(19) United States
(12) Patent Application Publication
(10) Pub. No.: US 2024/0276160 A1
(43) Pub. Date: Aug. 15, 2024

(54) AUDIO SIGNAL PROCESSING METHOD AND APPARATUS, ELECTRONIC DEVICE, AND COMPUTER-READABLE STORAGE MEDIUM

(71) Applicant: TENCENT TECHNOLOGY (SHENZHEN) COMPANY LIMITED, Shenzhen (CN)

(72) Inventors: Tingzhao WU, Shenzhen (CN); Wei XIAO, Shenzhen (CN); Yuyong KANG, Shenzhen (CN); Yupeng SHI, Shenzhen (CN); Shidong SHANG, Shenzhen (CN); Zurong WU, Shenzhen (CN)

(21) Appl. No.: 18/647,430

(22) Filed: Apr. 26, 2024

Related U.S. Application Data
(63) Continuation of application No. PCT/CN2023/090030, filed on Apr. 23, 2023.

(30) Foreign Application Priority Data
Jun. 30, 2022 (CN) 202210771358.9

(51) Int. Cl. H04R 25/00; A61B 5/12; G06F 3/16

(52) U.S. Cl. CPC H04R 25/505; A61B 5/123; G06F 3/165; H04R 25/558; H04R 25/70; H04R 2225/41; H04R 2225/43; H04R 2225/55; H04R 2430/01

(57) ABSTRACT

An audio signal processing method includes: displaying a hearing test control in a human-computer interaction interface; outputting a first test audio signal in response to a trigger operation on the hearing test control; displaying a first hearing test result of a target object in response to a feedback operation on the first test audio signal; and transmitting, to an audio device in response to a configuration operation on the audio device, a first hearing assistance policy generated according to the first hearing test result. The first hearing assistance policy is configured to be applied to the audio device to output a first audio signal adapted to the first hearing test result.

### CROSS-REFERENCES TO RELATED APPLICATIONS

This application is a continuation application of PCT Patent Application No. PCT/CN2023/090030, filed on Apr. 23, 2023, which claims priority to Chinese Patent Application No. 202210771358.9, filed on Jun. 30, 2022, both of which are incorporated herein by reference in their entirety.

### FIELD OF THE TECHNOLOGY

The present disclosure relates to the field of communication technologies, and in particular, to an audio signal processing method and apparatus, an electronic device, and a computer-readable storage medium.

### BACKGROUND

Hearing aids as professional equipment usually need face-to-face communication with audiologists in offline stores for fitting. For example, a fitting process with an audiologist may include as follows: The audiologist first needs to test hearing of a user, and then adjusts, according to a hearing test result by using a prescription formula, a parameter of a hearing aid worn by the user. As can be seen, such fitting process of the hearing aid is cumbersome, which leads to low efficiency.

### SUMMARY

Embodiments of the present disclosure provide an audio signal processing method and apparatus, an electronic device, a computer-readable storage medium, and a computer program product, which can implement efficient fitting of an audio device.

Technical solutions in the embodiments of the present disclosure are implemented as follows:

An embodiment of the present disclosure provides an audio signal processing method, including: displaying a hearing test control in a human-computer interaction interface; outputting a first test audio signal in response to a trigger operation on the hearing test control; displaying a first hearing test result of a target object in response to a feedback operation on the first test audio signal; and transmitting, to an audio device in response to a configuration operation on the audio device, a first hearing assistance policy generated according to the first hearing test result, where the first hearing assistance policy is configured to be applied to the audio device to output a first audio signal adapted to the first hearing test result.

An embodiment of the present disclosure provides an audio signal processing apparatus, including: a display module, configured to display a hearing test control in a human-computer interaction interface; an output module, configured to output a first test audio signal in response to a trigger operation on the hearing test control, the display module being further configured to display a first hearing test result of a target object in response to a feedback operation on the first test audio signal; and a transmitting module, configured to transmit, to an audio device in response to a configuration operation on the audio device, a first hearing assistance policy generated according to the first hearing test result, where the first hearing assistance policy is configured to be applied to the audio device to output a first audio signal adapted to the first hearing test result.

An embodiment of the present disclosure provides an audio signal processing method, including: obtaining a first hearing test result of a target object; determining, in descending order of frequencies of a plurality of sub-bands in an auditory frequency range, a filter parameter of each sub-band based on the first hearing test result, a filter parameter of a low-frequency sub-band being determined based on a filter parameter of a high-frequency sub-band; performing combination on the filter parameter of each sub-band, and using a combined filter bank parameter as a first hearing assistance policy for the target object; and transmitting the first hearing assistance policy to an audio device, where the first hearing assistance policy is configured to be applied to the audio device to output a first audio signal adapted to the first hearing test result.

An embodiment of the present disclosure provides an audio signal processing apparatus, including: an obtaining module, configured to obtain a first hearing test result of a target object; a determining module, configured to determine, in descending order of frequencies of each sub-band in an auditory frequency range, a filter parameter of each sub-band based on the first hearing test result, a filter parameter of a low-frequency sub-band being determined based on a filter parameter of a high-frequency sub-band; a combination module, configured to perform combination based on the filter parameter of each sub-band, and use an obtained filter bank parameter as a first hearing assistance policy for the target object; and a transmitting module, configured to transmit the first hearing assistance policy to an audio device, where the first hearing assistance policy is configured to be applied to the audio device to output a first audio signal adapted to the first hearing test result.

An embodiment of the present disclosure provides an audio signal processing method, including: receiving a first hearing assistance policy for a target object, the first hearing assistance policy including a filter bank parameter, the filter bank parameter including a filter parameter of a plurality of sub-bands in an auditory frequency range, the filter parameter of each sub-band being determined in descending order of frequencies based on a first hearing test result of the target object, and a filter parameter of a low-frequency sub-band being determined based on a filter parameter of a high-frequency sub-band; and outputting, according to the first hearing assistance policy, a first audio signal adapted to the first hearing test result.

An embodiment of the present disclosure provides an audio signal processing apparatus, including: a receiving module, configured to receive a first hearing assistance policy for a target object, the first hearing assistance policy including a filter bank parameter, the filter bank parameter including a filter parameter of each sub-band in an auditory frequency range, the filter parameter of each sub-band being determined in descending order of frequencies based on a first hearing test result of the target object, and a filter parameter of a low-frequency sub-band being determined based on a filter parameter of a high-frequency sub-band; and an output module, configured to output, according to the first hearing assistance policy, a first audio signal adapted to the first hearing test result.

An embodiment of the present disclosure provides an electronic device, including: a memory, configured to store an executable instruction; and a processor, configured to execute the executable instruction stored in the memory, to implement the audio signal processing method provided in the embodiments of the present disclosure.

An embodiment of the present disclosure provides a non-transitory computer-readable storage medium, storing an executable instruction, when executed by at least one processor, implementing the audio signal processing method provided in the embodiments of the present disclosure.

The embodiments of the present disclosure have the following beneficial effects:

A function of hearing testing and a function of configuring an audio device based on a hearing test result are integrated into a computer program, so that a user can configure the audio device through interaction with the computer program. Compared with a method in which the user needs to go to an offline store to configure the audio device, this lowers an operating threshold, improves efficiency in configuring the audio device, and improves auditory experience of the user.

### BRIEF DESCRIPTION OF THE DRAWINGS

FIG. 1 is a schematic architectural diagram of an audio signal processing system 100 according to an embodiment of the present disclosure.

FIG. 2A is a schematic structural diagram of a terminal device according to an embodiment of the present disclosure.

FIG. 2B is a schematic structural diagram of an audio device according to an embodiment of the present disclosure.

FIG. 3 is a schematic flowchart of an audio signal processing method according to an embodiment of the present disclosure.

FIG. 4 is a schematic flowchart of an audio signal processing method according to an embodiment of the present disclosure.

FIG. 5 is a schematic flowchart of an audio signal processing method according to an embodiment of the present disclosure.

FIG. 6 is a schematic diagram of function layout according to an embodiment of the present disclosure.

FIG. 7 is a schematic flowchart of pure tone hearing threshold and pain threshold testing according to an embodiment of the present disclosure.

FIG. 8 is a schematic flowchart of hearing threshold testing according to an embodiment of the present disclosure.

FIG. 9 is a schematic flowchart of pain threshold testing according to an embodiment of the present disclosure.

FIG. 10A to FIG. 10C are schematic diagrams of application scenarios of an audio signal processing method according to an embodiment of the present disclosure.

FIG. 11 is a schematic flowchart of pitch testing according to an embodiment of the present disclosure.

FIG. 12 is a schematic diagram of an application scenario of an audio signal processing method according to an embodiment of the present disclosure.

FIG. 13A is a schematic diagram of a frequency response curve corresponding to an offline fitting session.

FIG. 13B is a schematic diagram of a frequency response curve according to an embodiment of the present disclosure.

FIG. 14 is a schematic flowchart of personalized equalization according to an embodiment of the present disclosure.

FIG. 15 is a schematic flowchart of pitch adjustment according to an embodiment of the present disclosure.

FIG. 16 is a schematic diagram of an application scenario of an audio signal processing method according to an embodiment of the present disclosure.

FIG. 17 is a schematic flowchart of hearing sense adjustment according to an embodiment of the present disclosure.

FIG. 18 is a schematic diagram of an application scenario of an audio signal processing method according to an embodiment of the present disclosure.

### DESCRIPTION OF EMBODIMENTS

To make objectives, technical solutions, and advantages of the present disclosure clearer, the following describes the present disclosure in further detail with reference to accompanying drawings. The described embodiments are not to be considered as a limitation to the present disclosure. All other embodiments obtained by a person of ordinary skill in the art without creative efforts shall fall within the protection scope of the present disclosure.

In the following description, the term “some embodiments” describes subsets of all suitable embodiments, but it can be understood that “some embodiments” may be the same subset or different subsets of all the suitable embodiments, and can be combined with each other without conflict.

It can be understood that in the embodiments of the present disclosure, for related data such as user information (such as a hearing test result of a user), when the embodiments of the present disclosure are applied to a specific product or technology, permission or consent of the user needs to be obtained, and acquisition, use, and processing of the related data need to comply with related laws, regulations, and standards of related countries and regions.

In the following description, the terms “first/second/ . . . ” are merely for distinguishing between similar objects, and do not represent a specific order of objects. It can be understood that “first/second/ . . . ” may be interchanged where permitted, so that the embodiments of the present disclosure described herein can be implemented in an order other than that illustrated or described herein.

Unless otherwise defined, meanings of all technical and scientific terms used in this specification are the same as those usually understood by a person skilled in the art to which the present disclosure belongs. The terms used in this specification are merely for describing the embodiments of the present disclosure, but are not intended to limit the present disclosure.

Before the embodiments of the present disclosure are further described in detail, nouns and terms in the embodiments of the present disclosure are described, which are explained as follows:
(1) In response to: It is used for indicating a condition or state on which an operation to be performed depends. When the condition or state being depended on is met, one or more operations to be performed may be performed in real time or after a set delay. Unless specifically stated, the plurality of operations are not necessarily performed in a specific order.
(2) Hearing threshold: It is a minimal audible level, that is, intensity of a minimum sound that human ears can just hear, or minimum sound intensity for a person to distinguish existence of a sound.
(3) Pain threshold: It is intensity of a minimum sound that can cause physiological discomfort or pain in human ears.
(4) Sound pressure level (SPL): It is a physical quantity for describing sound pressure, defined as taking a common logarithm of a ratio of to-be-measured sound pressure p to reference sound pressure p(ref) and then multiplying the common logarithm by 20, in a unit of decibel (dB).
(5) Pitch: It is a sound frequency, which is one of three main subjective attributes of sounds, namely, volume (loudness), pitch, and timbre (also referred to as sound quality), and indicates a degree at which hearing of a person distinguishes a tone of a sound. Main pitches are limited, including, for example, a/a, i/yi, u/wu, m/mo, s/si, and sh/shi.
(6) Prescription formula: It is a formula for determining a gain value of each frequency band based on a hearing threshold of a target object in the frequency band, which is intended to provide a recommended gain for each hearing test frequency and input level. General prescription formulas include desired sensation level (DSL) and national acoustic laboratory (NAL) series. The DSL series formulas are intended to enable hearing aid wearers to obtain a maximum audible level in each frequency band. The NAL series formulas are intended to improve speech intelligibility while meeting hearing comfort of hearing-impaired people.
(7) Hearing assistance policy: It is a filter bank parameter obtained by combining filter parameters of a plurality of sub-bands in an auditory frequency range. A filter parameter of each sub-band is determined based on a hearing test result of a target object, and is applied to a hearing assistance device to assist in improving hearing of the target object.
(8) Target object: It is an object requiring hearing testing.

The embodiments of the present disclosure provide an audio signal processing method and apparatus, an electronic device, a computer-readable storage medium, and a computer program product, which can implement efficient and convenient configuration of an audio device. The following describes exemplary application of the electronic device provided in the embodiments of the present disclosure. The electronic device provided in the embodiments of the present disclosure may be implemented as various types of terminal devices such as a notebook computer, a tablet computer, a desktop computer, a set-top box, a mobile device (for example, a mobile phone, a portable music player, a personal digital assistant, a special messaging device, and a portable game device), and an in-vehicle terminal. Alternatively, the electronic device may be implemented as an audio device, or may be implemented by a terminal device and an audio device in cooperation. The audio device may be a power amplifier, a sound box, a multimedia console, a digital mixer, an audio sampling card, a synthesizer, a mid/high frequency speaker, a microphone, an audio card in a notebook computer, an earphone, a hearing aid, or other peripheral audio devices, such as a professional microphone series, a headset, and a public address system.

The following description is provided by using an example that a terminal device and an audio device cooperate to implement the audio signal processing method provided in the embodiments of the present disclosure.

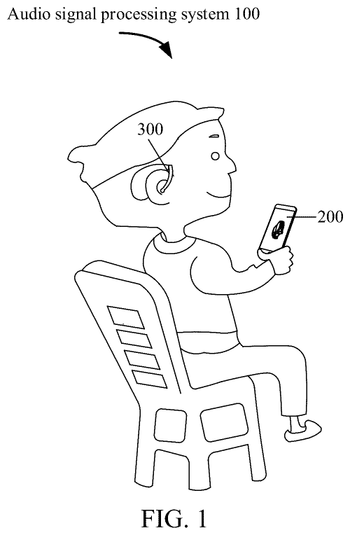

FIG. 1 is a schematic architectural diagram of an audio signal processing system 100 according to an embodiment of the present disclosure. To support an application (APP) that can efficiently and conveniently configure an audio device, as shown in FIG. 1, the audio signal processing system 100 includes: a terminal device 200 (for example, a mobile phone) and an audio device 300 (for example, a hearing aid). The terminal device 200 and the audio device 300 may be connected in a wired (for example, based on the universal serial bus (USB) protocol) or wireless (for example, based on the Bluetooth or ZigBee communication protocol) manner.

In some embodiments, a client (not shown in FIG. 1) runs on the terminal device 200. The client may be various types of clients, such as an instant messaging client, a network conference client, an audio-video playing client, a client dedicated for hearing testing, and a client configured for the audio device. A function of hearing testing and a function of configuring the audio device 300 based on a hearing test result are integrated into the client. In this way, a user can test hearing and configure the audio device based on a hearing test result through interaction with the client, which improves configuration efficiency, reduces operation costs of the user, and improves user experience.

The terminal device 200 may implement, by running a computer program, the audio signal processing method provided in the embodiments of the present disclosure. For example, the computer program may be a native program in an operating system or a software module; may be a native APP, that is, a program that needs to be installed in the operating system to run, for example, various types of clients such as a network conference APP, an instant messaging APP, and an audio-video playing APP; or may be an applet, that is, a program that only needs to be downloaded to a browser environment to run. In short, the computer program may be in any form such as an APP, a module, or a plug-in.

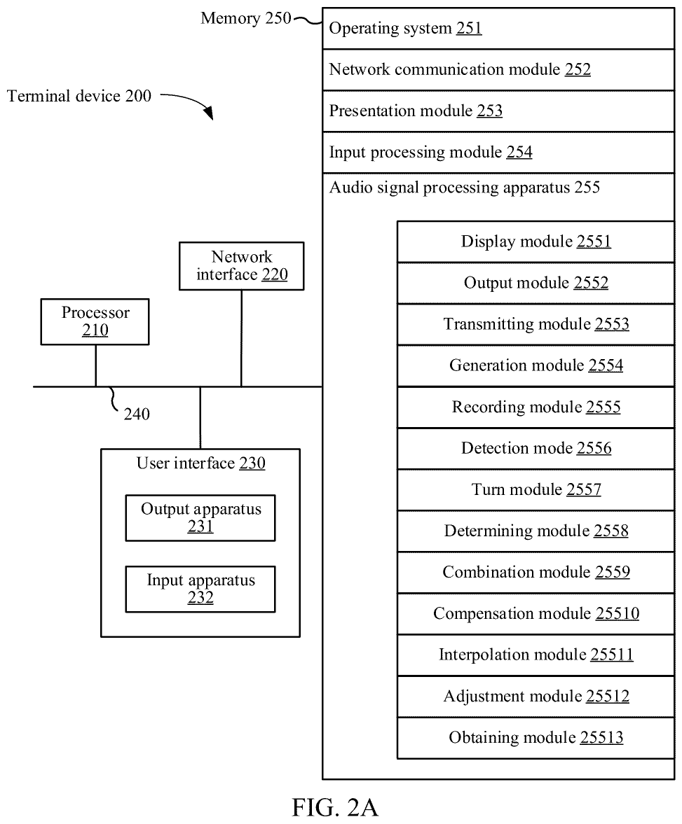

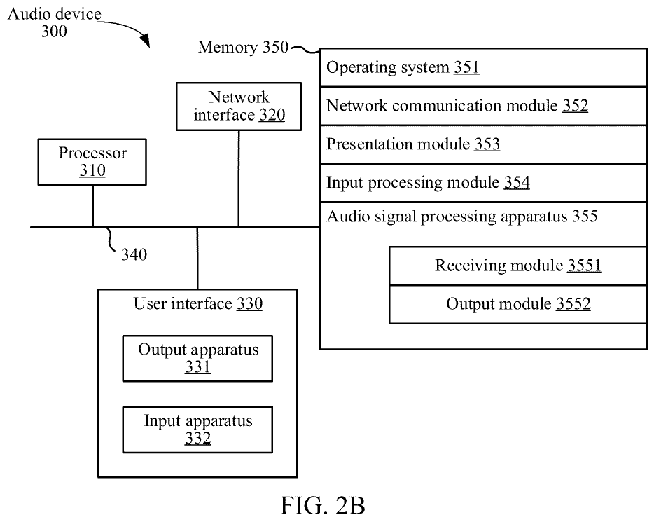

The following continues to describe a structure of the terminal device 200 shown in FIG. 1. FIG. 2A is a schematic structural diagram of the terminal device 200 according to an embodiment of the present disclosure. The terminal device 200 shown in FIG. 2A includes: at least one processor 210, a memory 250, at least one network interface 220, and a user interface 230. The components in the terminal device 200 are coupled through a bus system 240. It can be understood that, the bus system 240 is configured to implement connection and communication between the components. In addition to a data bus, the bus system 240 further includes a power bus, a control bus, and a status signal bus. However, for clear description, all the buses are marked as the bus system 240 in FIG. 2A.

The processor 210 may be an integrated circuit chip with a signal processing capability, such as a general-purpose processor, a digital signal processor (DSP) or another programmable logic device, a discrete gate, a transistor logic device, or a discrete hardware component. The general-purpose processor may be a microprocessor or any processor or the like.

The user interface 230 includes one or more output apparatuses 231 that can present media content, including one or more speakers and/or one or more visual displays. The user interface 230 further includes one or more input apparatuses 232, including user interface components that facilitate user inputting, such as a keyboard, a mouse, a microphone, a touchscreen, a camera, and other input buttons and controls.

The memory 250 may be a removable memory, a non-removable memory, or a combination thereof. Exemplary hardware devices include a solid-state memory, a hard disk drive, an optical disc drive, and the like. In some embodiments, the memory 250 includes one or more storage devices physically remote from the processor 210.

The memory 250 includes a volatile memory, a non-volatile memory, or both. The non-volatile memory may be a read-only memory (ROM). The volatile memory may be a random access memory (RAM). The memory 250 described in this embodiment of the present disclosure aims to include any memory of an appropriate type.

In some embodiments, the memory 250 can store data to support various operations. Examples of the data include programs, modules, and data structures or subsets or supersets thereof, as illustrated below.

An operating system 251 includes system programs for processing various basic system services and executing hardware-related tasks, for example, a framework layer, a kernel library layer, and a driver layer, to implement various basic services and process hardware-based tasks.

A network communication module 252 is configured to reach other computing devices via one or more (wired or wireless) network interfaces 220. Exemplary network interfaces 220 include: Bluetooth, wireless compatibility certification (Wi-Fi), USB, and other interfaces.

A presentation module 253 is configured to present information via one or more output apparatuses 231 (for example, a display and a speaker) associated with the user interface 230 (for example, a user interface for operating a peripheral device and displaying content and information).

An input processing module 254 is configured to detect one or more user inputs or interactions from one or more input apparatuses 232 and translate the detected input or interaction.

In some embodiments, the audio signal processing apparatus provided in the embodiments of the present disclosure may be implemented in a software manner. FIG. 2A shows an audio signal processing apparatus 255 stored in the memory 250, which may be software in a form of a program or a plug-in, including the following software modules: a display module 2551, an output module 2552, a transmitting module 2553, a generation module 2554, a recording module 2555, a detection module 2556, a turn module 2557, a determining module 2558, a combination module 2559, a compensation module 25510, an interpolation module 25511, an adjustment module 25512, and an obtaining module 25513. These modules are logical, and therefore may be arbitrarily combined or further split according to functions to implemented. The modules are all shown in FIG. 2A for ease of expression, but it is not to be interpreted that an implementation that may include only the display module 2551, the output module 2552, and the transmitting module 2553, or an implementation that may include only the obtaining module 25513, the determining module 2558, the combination module 2559, and the transmitting module 2553 is excluded in the audio signal processing apparatus 255. Functions of the modules are described below.

The following continues to describe a structure of the audio device 300 shown in FIG. 1. FIG. 2B is a schematic structural diagram of the audio device 300 according to an embodiment of the present disclosure. The audio device 300 shown in FIG. 2B includes: a processor 310, a network interface 320, a user interface 330 (including an output device 331 and an input device 332), a bus system 340, and a memory 350. The memory 350 includes: an operating system 351, a network communication module 352, a presentation module 353, an input processing module 354, and an audio signal processing apparatus 355. In addition, the audio signal processing apparatus 355 stored in the memory 350 may be software in a form of a program or a plug-in, including the following software modules: a receiving module 3551 and an output module 3552. These modules are logical, and therefore may be arbitrarily combined or further split according to functions to implemented. Functions of the modules are described below. In addition, the functions of the components in FIG. 2B are similar to those of corresponding components in FIG. 2A, reference may be made to the description of FIG. 2A, and details are not described herein again in this embodiment of the present disclosure.

The audio signal processing method provided in the embodiments of the present disclosure is specifically described below from the perspective of interaction between the terminal device and the audio device.

Steps performed by the terminal device are specifically performed by various forms of computer programs running on the terminal device, which are not limited to a client, but may alternatively be an operating system, a software module, or a script described above. Therefore, the client is not to be considered as a limitation on the embodiments of the present disclosure. In addition, for ease of description, no specific distinction is made below between the terminal device and the computer program running on the terminal device.

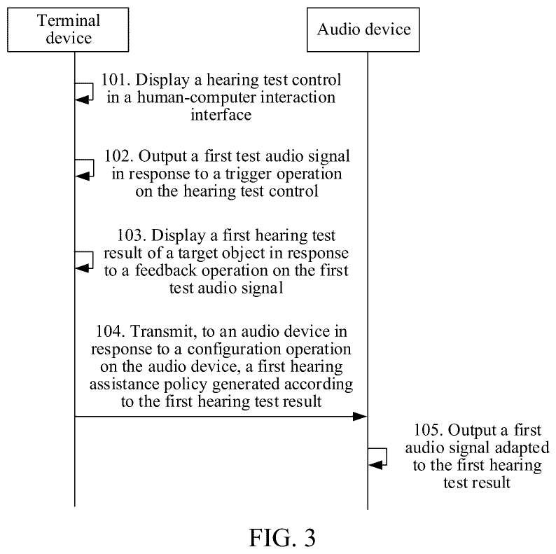

FIG. 3 is a schematic flowchart of an audio signal processing method according to an embodiment of the present disclosure. The method is described with reference to steps shown in FIG. 3.

In step 101, a terminal device displays a hearing test control in a human-computer interaction interface.

In some embodiments, a client runs on a terminal device associated with a target object (that is, an object requiring hearing testing, for example, a user A), and a hearing test control, for example, a “start test” button, is displayed in a human-computer interaction interface provided by the client.

In some other embodiments, the terminal device may further perform the following processing before displaying the hearing test control in the human-computer interaction interface: displaying a historical hearing test result of the target object in the human-computer interaction interface in response to existence of the historical hearing test result and the historical hearing test result being within a validity period (for example, 3 months); and transmitting, to an audio device in response to a configuration operation on the audio device, a fourth hearing assistance policy generated according to the historical hearing test result, the fourth hearing assistance policy being applied to the audio device to output a fourth audio signal adapted to the historical hearing test result. This can save time required for the user to test hearing, and further improve efficiency in configuring the audio device.

For example, the validity period is a maximum interval between current time and the last time of testing. The validity period may be manually preset. For example, the validity period may be set to 3 months or half a year.

For example, the historical hearing test result may be a historical hearing test result of the target object obtained from a third-party hearing detection organization, or may be a historical hearing test result that is obtained from hearing testing previously performed by the target object based on an APP and that is obtained from the terminal device locally or a server.

In step 102, the terminal device outputs a first test audio signal in response to a trigger operation on the hearing test control.

In some embodiments, when the terminal device receives a trigger operation of the target object on the hearing test control (for example, the “start test” button) displayed in the human-computer interaction interface, the terminal device may obtain a first test audio signal from a server, or invoke a computing capability of the terminal device to generate a first test audio signal locally in the terminal device based on factors such as a channel, a frequency, and a sound pressure level (SPL), or obtain a first test audio signal from a plurality of test audio signals stored in advance in the terminal device locally, and transmit the first test audio signal to an audio apparatus (for example, a speaker) disposed in the terminal device, and the audio apparatus outputs the first test audio signal. Certainly, the terminal device may alternatively transmit the first test audio signal to an external audio device, and the audio device outputs the first test audio signal.

In some other embodiments, before outputting the first test audio signal, the terminal device may further detect an SPL of an environment in which the target object is located. The terminal device turns to the operation of outputting a first test audio signal in a case that an average SPL of the environment in which the target object is located within a set duration (for example, 2 seconds) is less than an SPL threshold (for example, 40 dB). In this way, before hearing testing, an environment is first detected to ensure that the target object is in a relatively quiet environment, thereby improving accuracy of a subsequent hearing test result.

The SPL threshold may be an average SPL value of environments assessed as quiet environments by a plurality of tested persons. For example, five persons are tested. Assuming that a tested person 1 assesses an environment at an SPL less than 42 dB as a quiet environment, a tested person 2 assesses an environment at an SPL less than 38 dB as a quiet environment, a tested person 3 assesses an environment at an SPL less than 41 dB as a quiet environment, a tested person 4 assesses an environment at an SPL less than 39 dB as a quiet environment, and a tested person 5 assesses an environment at an SPL less than 40 dB as a quiet environment, an average value (that is, 40 dB) of these five SPLs may be used as the SPL threshold.

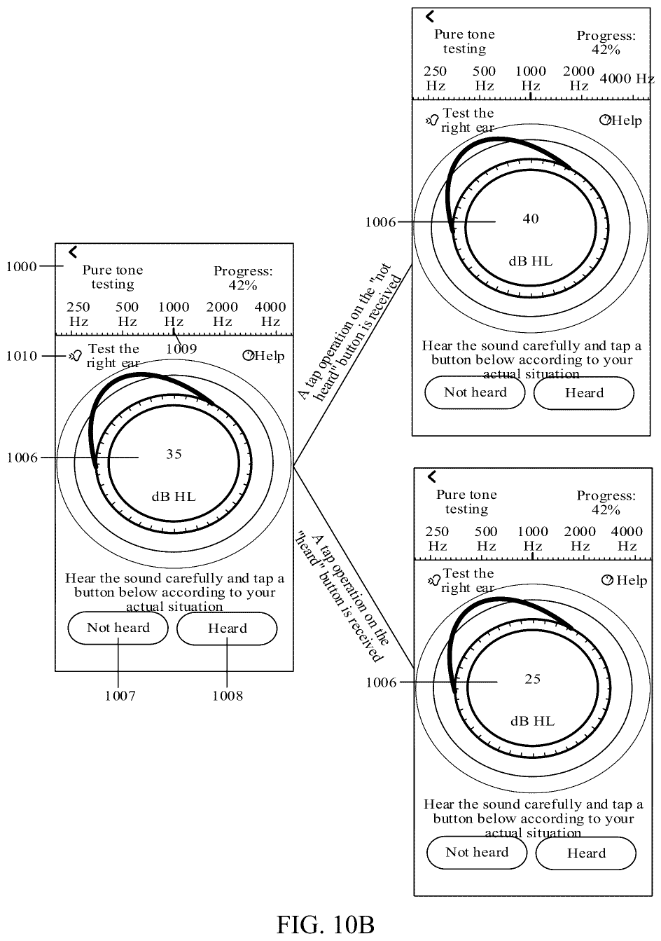

For example, FIG. 10A is a schematic diagram of an application scenario of an audio signal processing method according to an embodiment of the present disclosure. As shown in FIG. 10A, a hearing test control, for example, a “start test” button 1001, is displayed in a human-computer interaction interface 1000. In addition, three detection controls are also displayed in the human-computer interaction interface 1000: a “select a quiet environment” control 1002 for detecting whether the environment in which the target object is located meets a hearing test requirement, a “put on an earphone” control 1003 for detecting whether the target object has put on an earphone, and a “turn your mobile phone to a comfortable volume” control 1004 for detecting whether a volume of current output of the mobile phone is appropriate.

Before the three detection steps are completed, the “start test” button 1001 may be in a disabled state. For example, the “start test” button 1001 may be displayed in a gray mode, and a response to a tap operation on the “start test” button 1001 may be shielded. That is, before the detection steps are completed, the user cannot test hearing, thereby ensuring accuracy of subsequent hearing testing. Certainly, the user may alternatively directly test hearing by tapping a “direct test” button 1005 displayed in the human-computer interaction interface 1000, to save time of the user.

In step 103, the terminal device displays a first hearing test result of the target object in response to a feedback operation on the first test audio signal.

In some embodiments, the first hearing test result may include at least one of a hearing parameter and a speech recognition capability parameter, and the first test audio signal may include at least one of the following types of test audio signals: a hearing test audio signal for testing hearing of the target object, and a speech recognition capability test audio signal for testing a speech recognition capability of the target object. Then, the terminal device may perform step 103 in the following manner: generating a hearing parameter of the target object in response to a feedback operation on the hearing test audio signal; generating a speech recognition capability parameter of the target object in response to a feedback operation on the speech recognition capability test audio signal; and displaying a hearing test result including at least one of the hearing parameter and the speech recognition capability parameter.

In some other embodiments, further, the hearing parameter may include a hearing threshold of the target object in each sub-band in an auditory frequency range. For example, the auditory frequency range may be divided into six sub-bands respectively having center frequencies of 250 Hz, 500 Hz, 1000 Hz, 2000 Hz, 4000 Hz, and 8000 Hz according to response characteristics of a human ear to different frequencies. Then, the hearing parameter of the target object may be generated in response to the feedback operation on the hearing test audio signal in the following manner: performing the following processing for any sub-band in the auditory frequency range: displaying an SPL control (configured to indicate an SPL of a currently outputted hearing test audio signal) and the following feedback controls in the human-computer interaction interface: a first feedback control (for example, a “not heard” button) configured to indicate that the hearing test audio signal is not heard by the target object, and a second feedback control (for example, a “heard” button) configured to indicate that the hearing test audio signal is heard by the target object; re-outputting the hearing test audio signal (the hearing test audio signal has a specific duration) in a manner higher than an SPL of current output in response to a trigger operation on the first feedback control; re-outputting the hearing test audio signal in a manner lower than an SPL of current output in response to a trigger operation on the second feedback control; and for any SPL used for current output, determining the any SPL as a hearing threshold of the target object in the sub-band in response to receiving a second trigger operation on the second feedback control at the any SPL.

For example, FIG. 10B is a schematic diagram of an application scenario of an audio signal processing method according to an embodiment of the present disclosure. As shown in FIG. 10B, an SPL control 1006 configured to indicate an SPL (for example, 35 dB) of a currently outputted hearing test audio signal, a first feedback control (for example, a “not heard” button 1007), and a second feedback control (for example, a “heard” button 1008) are displayed in a human-computer interaction interface 1000. In addition, a value 1009 (for example, 1000 Hz) of a center frequency of a current sub-band and prompt information 1010 (for example, test the right ear) of a currently tested ear are also displayed in the human-computer interaction interface 1000.

Still refer to FIG. 10B. When a tap operation of the target object on the “not heard” button 1007 is received, the hearing test audio signal is re-outputted in a manner (for example, 40 dB) higher than the SPL of the current output, and the value 1006 of the SPL of the current output displayed in the human-computer interaction interface 1000 is updated from 35 dB to 40 dB. When a tap operation of the target object on the “heard” button 1008 is received, the hearing test audio signal is re-outputted in a manner (for example, 25 dB) lower than the SPL of the current output, and the value 1006 of the SPL of the current output displayed in the human-computer interaction interface 1000 is updated from 35 dB to 25 dB. This is repeated, and when a second tap of the target object on the “heard” button 1008 at an SPL is received, the current SPL is recorded as a hearing threshold of the target object in the current sub-band.

The sub-band with the center frequency of 1000 Hz is used as an example for description. The hearing test audio signal is outputted first at an SPL of 30 dB. If a tap operation of the user A on the “heard” button 1008 is received in this case, the SPL of the hearing test audio signal is reduced by 10 dB, that is, the hearing test audio signal is outputted at an SPL of 20 dB. If a tap operation of the user A on the “not heard” button 1007 is received at the SPL of 20 dB, the SPL of the hearing test audio signal is increased by 5 dB, that is, the hearing test audio signal is outputted at an SPL of 25 dB. If a tap operation of the user A on the “not heard” button 1007 is received at the SPL of 25 dB, the SPL of the hearing test audio signal is further increased by 5 dB, that is, the hearing test audio signal is outputted at the SPL of 30 dB. If a tap operation of the user A on the “heard” button 1008 is received again at the SPL of 30 dB, 30 dB may be used as a hearing threshold of the user A in the sub-band with the center frequency of 1000 Hz.

In some other embodiments, the following processing may alternatively be performed for any sub-band in the auditory frequency range: for any SPL used for current output, determining the any SPL as a hearing threshold of the target object in the sub-band in response to receiving a trigger operation of the target object on the second feedback control at the any SPL.

The sub-band with the center frequency of 1000 Hz is used as an example. The hearing test audio signal is sequentially outputted at different SPLs by continuously increasing an SPL. For example, the hearing test audio signal is outputted first at an SPL of 20 dB. If a tap operation of the user A on the “not heard” button 1007 is received in this case, the SPL of the hearing test audio signal is increased by 5 dB, that is, the hearing test audio signal is outputted at an SPL of 25 dB. If a tap operation of the user A on the “not heard” button 1007 is still received at the SPL of 25 dB, the SPL of the hearing test audio signal is further increased by 5 dB, that is, the hearing test audio signal is outputted at an SPL of 30 dB. If a tap operation of the user A on the “heard” button 1008 is received at the SPL of 30 dB, 30 dB may be used as a hearing threshold of the user A in the sub-band with the center frequency of 1000 Hz. This simplifies a process of hearing threshold testing, thereby saving time of the user.

The hearing threshold is not a fixed value, that is, it does not mean that the user absolutely can hear a sound at an SPL, and absolutely cannot hear a sound below this SPL. In fact, it is a gradual transition process from “not heard” to “sometimes heard and sometimes not heard” and to “heard” as sound intensity increases. Therefore, a plurality of hearing threshold tests may be performed for the target object, and an average value of hearing thresholds obtained from the plurality of hearing threshold tests is used as a hearing threshold of the target object, to further improve accuracy of a testing result.

In some embodiments, the hearing parameter may further include a pain threshold of the target object in each sub-band in the auditory frequency range. Then, the hearing parameter of the target object may be generated in response to the feedback operation on the hearing test audio signal in the following manner: performing the following processing for any sub-band in the auditory frequency range: displaying an SPL control (configured to indicate an SPL of a currently outputted hearing test audio signal), a first adjustment control (for example, a slider bar), and a third feedback control (for example, an “ear uncomfortable” button) in the human-computer interaction interface, the third feedback control being configured to indicate that physiological discomfort occurs on the target object in hearing the hearing test audio signal; adjusting the SPL of the currently outputted hearing test audio signal in response to a trigger operation on the first adjustment control; and in response to a trigger operation on the third feedback control, determining an SPL in response to that the trigger operation is received as a pain threshold of the target object in the sub-band.

For example, FIG. 10C is a schematic diagram of an application scenario of an audio signal processing method according to an embodiment of the present disclosure. As shown in FIG. 10C, an SPL control 1011 configured to indicate an SPL (for example, 77 dB) of a currently outputted hearing test audio signal, a first adjustment control, for example, a slider bar 1012, an adjustment button 1013 being displayed on the slider bar 1012 so that the user may adjust the SPL of the currently outputted hearing test audio signal by sliding the adjustment button 1013, and a third feedback control, for example, an “ear uncomfortable” button 1014, are displayed in a human-computer interaction interface 1000. In addition, a value 1015 (for example, 2000 Hz) of a center frequency of a current sub-band and prompt information 1016 (for example, test the right ear) of a currently tested ear are also displayed in the human-computer interaction interface 1000. For example, assuming that a tap operation of the target object (for example, the user A) on the “ear uncomfortable” button 1014 displayed in the human-computer interaction interface 1000 is received when the SPL of the outputted hearing test audio signal is 80 dB, 80 dB may be determined as a pain threshold of the user A in the sub-band with the center frequency of 2000 Hz.

In some embodiments, the terminal device may further generate the speech recognition capability parameter of the target object in response to the feedback operation on the speech recognition capability test audio signal in the following manner: displaying a decibel control (configured to indicate a decibel value of a currently outputted speech recognition capability test audio signal) and a plurality of fourth feedback controls in the human-computer interaction interface, each fourth feedback control being corresponding to a pitch; sequentially outputting a plurality of speech recognition capability test audio signals, and recording a fourth feedback control triggered by the target object in the plurality of fourth feedback controls each time the speech recognition capability test audio signal is outputted; and determining a correctness percentage of pitch recognition of the target object based on pitches respectively corresponding to the plurality of speech recognition capability test audio signals and fourth feedback controls respectively triggered by the target object in a plurality of test processes. That is, each time the speech recognition capability test audio signal is outputted, it is determined whether a pitch corresponding to the speech recognition capability test audio signal outputted by the audio device is consistent with a pitch corresponding to the fourth feedback control triggered by the target object. When the pitches are consistent, it is determined that the target object succeeds in pitch recognition, when the pitches are inconsistent, it is determined that the target object fails in pitch recognition, and the determined correctness percentage of pitch recognition is used as the speech recognition capability parameter of the target object.

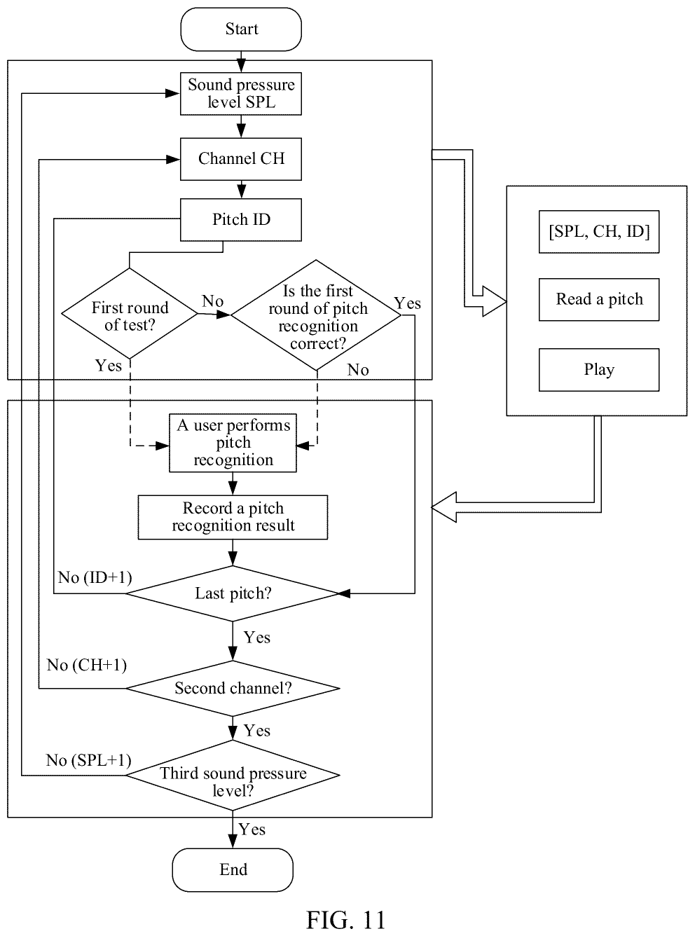

For example, FIG. 12 is a schematic diagram of an application scenario of an audio signal processing method according to an embodiment of the present disclosure. As shown in FIG. 12, a decibel control 1201 configured to indicate a decibel value (for example, 50 dB) of a currently outputted speech recognition capability test audio signal and a plurality of fourth feedback controls, each fourth feedback control being corresponding to a pitch, for example, including an “a/a” button 1202, a “m/mo” button 1203, an “i/yi” button 1204, a “s/si” button 1205, a “u/wu” button 1206, and a “sh/shi” button 1207, are displayed in a human-computer interaction interface 1200. In addition, an “inaudible” button 1208 is also displayed in the human-computer interaction interface 1200. When a tap operation of the target object on the “inaudible” button 1208 is received, the speech recognition capability test audio signal may be re-outputted, or the speech recognition capability test audio signal may be re-outputted in a manner higher than a current decibel value. In addition, prompt information 1209 (for example, test the right ear) of a currently tested ear is also displayed in the human-computer interaction interface 1200.

For example, the target object is the user A. It is assumed that 10 speech recognition capability test audio signals are sequentially outputted to the user A, pitches respectively corresponding to the 10 speech recognition capability test audio signals are: u/wu, s/si, i/yi, sh/shi, a/a, u/wu, s/si, m/mo, u/wu, i/yi, and fourth feedback controls respectively triggered by the user A in these 10 test processes are: the “u/wu” button 1206, the “sh/shi” button 1207, the “i/yi” button 1204, the “s/si” button 1205, the “a/a” button 1202, “u/wu” button 1206, the “sh/shi” button 1207, the “m/mo” button 1203, the “u/wu” button 1206, and the “i/yi” button 1204. The user A recognizes three pitches incorrectly. Then, it may be determined that a correctness percentage of pitch recognition of the user A is 70%, and the correctness percentage 70% is used as a speech recognition capability parameter of the user A.

In step 104, the terminal device transmits, to the audio device in response to a configuration operation on the audio device, a first hearing assistance policy generated according to the first hearing test result.

In some embodiments, the terminal device may further perform the following processing before transmitting, to the audio device, the first hearing assistance policy generated according to the first hearing test result: determining, in descending order of frequencies, a filter parameter of each sub-band in the auditory frequency range based on the first hearing test result; and performing combination based on the filter parameter of each sub-band, and using an obtained filter bank parameter as the first hearing assistance policy for the target object.

For example, the first hearing test result may include the hearing threshold of the target object in each sub-band in the auditory frequency range. Then, the terminal device may determine, in descending order of frequencies, the filter parameter of each sub-band in the auditory frequency range based on the first hearing test result in the following manner: obtaining a gain value of each sub-band based on the hearing threshold of the target object in each sub-band and a prescription formula (for example, the NAL series prescription formulas or the DSL series prescription formula), for example, the hearing threshold of the target object in each sub-band may be substituted into the prescription formula for calculation to obtain the gain value of the corresponding sub-band; and obtaining, in descending order of frequencies, the filter parameter of each sub-band based on the gain value of each sub-band. In this way, the filter parameter is determined by using a “backward” calculation method, that is, a filter parameter corresponding to a high-frequency sub-band is determined first, and then a filter parameter of a low-frequency sub-band is calculated according to a characteristic of a frequency response that undergoes filtering, so that a desired frequency response curve can be closer, thereby achieving a better gain effect and improving auditory experience of the user.

For example, the auditory frequency range includes N sub-bands (for example, six sub-bands). It is assumed that a sixth sub-band is a sub-band with a center frequency of 8000 Hz, a fifth sub-band is a sub-band with a center frequency of 4000 Hz, a fourth sub-band is a sub-band with a center frequency of 2000 Hz, a third sub-band is a sub-band with a center frequency of 1000 Hz, a second sub-band is a sub-band with a center frequency of 500 Hz, and a first sub-band is a sub-band with a center frequency of 250 Hz, N being an integer greater than 1. Then, the terminal device may obtain, in descending order of frequencies, the filter parameter of each sub-band based on the gain value of each sub-band in the following manner: substituting a gain value of the Ni sub-band into a filter function for calculation to obtain a filter parameter of the Ni sub-band; and determining a filter parameter of an i^{th} sub-band based on a difference between a gain value of the i^{th} sub-band and a frequency response of a filter of an (i+1)^{th} sub-band at the i^{th} sub-band. For example, a filter parameter of the sixth sub-band is first calculated based on a gain value of the sixth sub-band, a filter parameter of the fifth sub-band is then calculated based on a difference between a gain value of the fifth sub-band and a frequency response of a filter of the sixth sub-band at the fifth sub-band, and so on, so that filter parameters respectively corresponding to the six sub-bands can be obtained. A value range of i meets 1≤i≤N−1, and a frequency of the (i+1)^{th} sub-band is greater than a frequency of the i^{th} sub-band.

The first hearing assistance policy may be generated in real time in response to the configuration operation triggered by the target object, or may be generated in advance; and may be generated locally in the terminal device, or may be generated in a server. For example, the terminal device sends the first hearing test result of the target object to the server, and the server generates the first hearing assistance policy. This is not specifically limited in this embodiment of the present disclosure.

In step 105, the audio device outputs a first audio signal adapted to the first hearing test result.

In some embodiments, the audio device may output, in the following manner, the first audio signal adapted to the first hearing test result: controlling, in ascending order of frequencies, a filter of each sub-band in a filter bank to sequentially filter an original audio signal according to a filter parameter of the corresponding sub-band in the filter bank parameter, to obtain the first audio signal adapted to the first hearing test result.

For example, the filter bank parameter is obtained by combining filter parameters of six sub-bands. After the audio device receives the original audio signal, the original audio signal may be filtered by filters of the six sub-bands in ascending order of frequencies, that is, sequentially processed by the six filters from a low frequency to a high frequency, to obtain the first audio signal adapted to the first hearing test result (that is, an audio signal that undergoes personalized equalization). In addition, to prevent a “clipping” phenomenon from occurring in the first audio signal finally outputted and affecting a hearing sense of the target object, before the first audio signal is outputted, dynamic range control (DRC) may be further performed to ensure integrity of the first audio signal.

The DRC means mapping a dynamic range of an input audio signal to a specified dynamic range. The dynamic range after the mapping is usually smaller than the dynamic range before the mapping. Therefore, the DRC is also referred to as dynamic range compression. The DRC provides compression and amplification capabilities, so that a sound can sound softer or louder, which is a signal amplitude adjustment method.

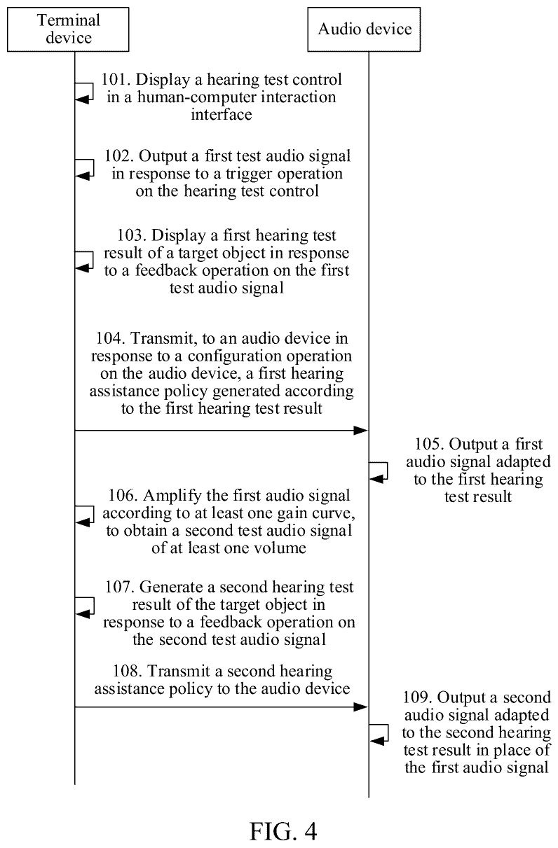

In some other embodiments, FIG. 4 is a schematic flowchart of an audio signal processing method according to an embodiment of the present disclosure. As shown in FIG. 4, steps 106 to 109 shown in FIG. 4 may be performed after step 105 shown in FIG. 3 is performed. The method is described with reference to steps shown in FIG. 4.

In step 106, the terminal device amplifies the first audio signal according to at least one gain curve, to obtain a second test audio signal of at least one volume.

In some embodiments, the terminal device may further perform the following processing before amplifying the first audio signal according to the at least one gain curve: obtaining characteristic information (for example, an age, a wearing side, and wearing years) of the target object; determining a gain factor of the first audio signal according to the characteristic information of the target object; generating the at least one gain curve according to the hearing parameter (including at least one of the hearing threshold and the pain threshold of the target object in each sub-band in the auditory frequency range) included in the first hearing test result, the gain factor, and the prescription formula, each gain curve being corresponding to a volume, for example, three gain curves may be calculated according to the gain factor, the hearing threshold, and the pain threshold by using the prescription formula, and respectively correspond to a plurality of volumes, including low sound, medium sound, and loud sound, where the plurality of volumes may be obtained through uniform or non-uniform division according to a decibel range of sounds that can be perceived by humans, for example, a decibel value being 0 dB to 20 dB may be defined as low sound, a decibel value being 20 dB to 60 dB may be defined as medium sound, and a decibel value greater than 60 dB may be defined as loud sound; and then performing interpolation processing on each gain curve through frequency band mapping, so that the number of sub-bands of the gain curve is consistent with the number of channels of the filter bank.

For example, when the sub-bands of the gain curve are mapped to the channels of the filter bank, because the number of sub-bands of the gain curve is less than the number of channels of the filter bank, for example, it is assumed that the original number of sub-bands of the gain curve is 5 while the number of channels of the filter bank is 8, interpolation processing needs to be performed on the gain curve. For example, interpolation processing may be performed on the gain curve through linear interpolation or parabolic interpolation, so that the number of sub-bands of the gain curve that undergoes the interpolation processing is increased to 8.

In step 107, the terminal device generates a second hearing test result of the target object in response to a feedback operation on the second test audio signal.

In some embodiments, the terminal device may perform step 107 in the following manner: displaying a second adjustment control (for example, a slider bar), a plurality of fifth feedback controls, and a plurality of volume controls in the human-computer interaction interface, each fifth feedback control being corresponding to a pitch, and a volume represented by a volume control in a selected state being used as a volume used during outputting of the second test audio signal; adjusting a gain of the currently outputted second test audio signal in response to a trigger operation on the second adjustment control; sequentially outputting a plurality of second test audio signals, and recording a fifth feedback control triggered by the target object in the plurality of fifth feedback controls each time the second test audio signal is outputted; and obtaining, based on pitches respectively corresponding to the plurality of second test audio signals and fifth feedback controls respectively triggered by the target object in a plurality of test processes, a pitch incorrectly recognized by the target object. For example, each time the second test audio signal is outputted, it is determined whether a pitch corresponding to the second test audio signal is consistent with a pitch corresponding to the fifth feedback control triggered by the target object. When the pitches are inconsistent, the pitch corresponding to the second test audio signal is determined as the pitch incorrectly recognized by the target object, and the pitch incorrectly recognized by the target object is used as the second hearing test result of the target object.

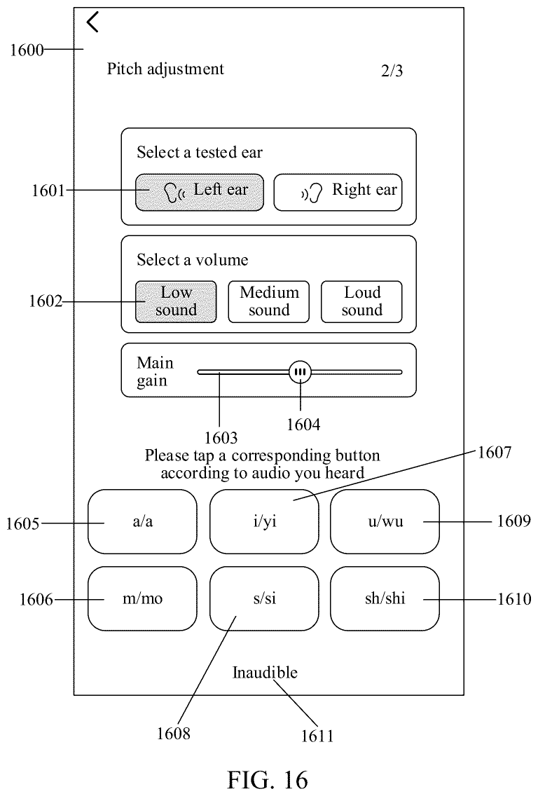

For example, FIG. 16 is a schematic diagram of an application scenario of an audio signal processing method according to an embodiment of the present disclosure. As shown in FIG. 16, in a human-computer interaction interface 1600, a tested ear is highlighted, for example, assuming that a left ear is currently tested, a left ear control 1601 may be highlighted; and a selected volume is highlighted, for example, assuming that a currently selected volume is low sound, a low sound control 1602 may be highlighted. In addition, a second adjustment control, for example, a slider bar 1603, an adjustment button 1604 being displayed on the slider bar 1603 so that the user may adjust a main gain (that is, a gain of a currently outputted second test audio signal) by sliding the adjustment button 1604, and a plurality of fifth feedback controls, each fifth feedback control being corresponding to a pitch, for example, including an “a/a” button 1605, a “m/mo” button 1606, an “i/yi” button 1607, a “s/si” button 1608, a “u/wu” button 1609, and a “sh/shi” button 1610, are also displayed in the human-computer interaction interface 1600. In this way, a plurality of second test audio signals are outputted to the target object (for example, the user A), and a fifth feedback control triggered by the user A each time the user A hears a second test audio signal is recorded, to obtain a pitch incorrectly recognized by the user A (that is, the second hearing test result).

In addition, an “inaudible” button 1611 is also displayed in the human-computer interaction interface 1600. When a tap operation of the target object on the “inaudible” button 1611 is received, the second test audio signal may be re-outputted, or the second test audio signal may be re-outputted in a manner higher than a current decibel value.

In step 108, the terminal device transmits a second hearing assistance policy to the audio device.

In some embodiments, the second hearing assistance policy may be obtained by adjusting the first hearing assistance policy according to the second hearing test result. The second hearing test result includes the pitch incorrectly recognized by the target object. Then, the terminal device may further perform the following processing before transmitting the second hearing assistance policy to the audio device: pertinently compensating the first hearing assistance policy according to the pitch incorrectly recognized by the target object, to obtain the second hearing assistance policy, that is, adding, according to the pitch incorrectly recognized by the target object, an adjustment amount of the pitch incorrectly recognized by the target object in the first hearing assistance policy, to obtain the second hearing assistance policy.

For example, a specific process of the pertinent compensation may be: for the pitch incorrectly recognized by the target object, compensating, according to a frequency corresponding to the pitch, a filter parameter of a corresponding sub-band in the filter bank parameter included in the first hearing assistance policy. For example, a filter corresponding to the frequency may be determined from the filter bank according to the frequency corresponding to the pitch, and then a parameter of the filter is compensated. For example, it is assumed that the frequency corresponding to the pitch is 500 Hz. As the center frequency of the third sub-band is exactly 500 Hz, it may be determined that a filter parameter of the third sub-band in the filter bank needs to be compensated, that is, a specific adjustment amount is added so that the target object can perceive the pitch as a compensation target.

The adjustment amount may be fixed or dynamic. For example, the adjustment amount may be a fixed value preset by operation personnel of the APP, that is, a fixed adjustment amount is added each time. Certainly, the adjustment amount may alternatively be dynamically changed according to different error situations, that is, a different adjustment amount may be added each time.

In an example of compensation, assuming that the pitch incorrectly recognized by the target object is “sh/shi”, a filter parameter of a corresponding sub-band in the filter bank parameter included in the first hearing assistance policy may be compensated pertinently according to the pitch “sh/shi” incorrectly recognized by the target object. For example, a volume of the pitch “sh/shi” may be increased so that the target object can clearly hear the pitch. In addition, different error situations may correspond to different compensation, while an adjustment amount of the compensation may be preset without manual adjustment of the user. In step 109, the audio device outputs a second audio signal adapted to the second hearing test result in place of the first audio signal.

In some embodiments, after receiving the second hearing assistance policy transmitted by the terminal device, the audio device may use the second hearing assistance policy in place of the first hearing assistance policy received in step 104. In this way, when an original audio signal is subsequently received, the received original audio signal may be adjusted by using the second hearing assistance policy. For example, the original audio signal may be sequentially filtered from a low frequency to a high frequency based on the pertinently compensated filter bank parameter included in the second hearing assistance policy, to output a second audio signal adapted to the second hearing test result (that is, an audio signal obtained through pitch adjustment based on the first audio signal), thereby further improving auditory experience of the user.

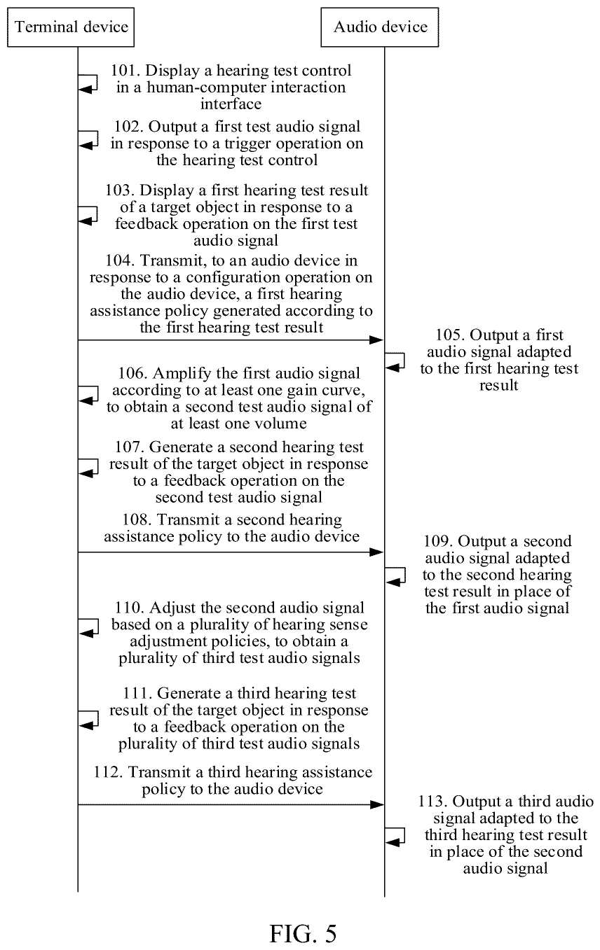

In some embodiments, FIG. 5 is a schematic flowchart of an audio signal processing method according to an embodiment of the present disclosure. As shown in FIG. 5, steps 110 to 113 shown in FIG. 5 may be performed after step 109 shown in FIG. 4 is performed. The method is described with reference to steps shown in FIG. 5.

In step 110, the terminal device adjusts the second audio signal based on a plurality of candidate hearing sense adjustment policies, to obtain a plurality of third test audio signals.

In some embodiments, the audio device may not output the second audio signal, but directly output a third audio signal obtained by adjusting a hearing sense of the second audio signal. For example, a plurality of different types of candidate hearing sense adjustment policies may be displayed in the human-computer interaction interface of the terminal device for the target object to select. Then, the terminal device may adjust the hearing sense of the second audio signal based on a plurality of hearing sense adjustment policies selected by the target object to obtain a plurality of third test audio signals.

In an example in which the terminal device adjusts the hearing sense of the second audio signal based on a hearing sense adjustment policy, a pitch carried in the hearing sense adjustment policy is first obtained, and then the second audio signal is adjusted through wide dynamic range compression based on a frequency corresponding to the obtained pitch, to obtain a third test audio signal. For example, a frequency of the second audio signal may be reduced, and a corresponding gain value may be adjusted in real time according to sound intensity (for example, a decibel value) of the second audio signal, so that the third test audio signal finally obtained sounds lower than the second audio signal. In addition, each third test audio signal corresponds to a different hearing sense. For example, the hearing sense of the second audio signal may be adjusted by using four different types of hearing sense adjustment policies, to obtain third test audio signals with four different hearing senses: an original hearing sense, higher, lower, and a clearer speech.

The wide dynamic range compression means that as sound intensity of an input audio signal changes, a corresponding gain also changes in real time, so that an amplified audio signal completely falls within a reduced auditory dynamic range of a hearing-impaired user.

In step 111, the terminal device generates a third hearing test result of the target object in response to a feedback operation on the plurality of third test audio signals.

In some embodiments, the third hearing test result may include a preferred hearing sense of the target object. Then, the terminal device may perform step 111 in the following manner: displaying a plurality of sixth feedback controls in the human-computer interaction interface, each sixth feedback control being corresponding to a hearing sense; and sequentially outputting a plurality of third test audio signals in one-to-one correspondence with the plurality of sixth feedback controls, and determining a hearing sense corresponding to a sixth feedback control triggered by the target object in the plurality of sixth feedback controls as the preferred hearing sense of the target object.

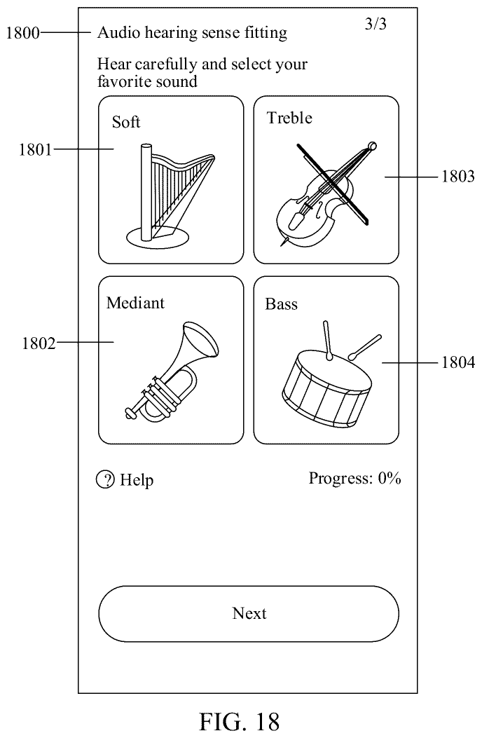

For example, FIG. 18 is a schematic diagram of an application scenario of an audio signal processing method according to an embodiment of the present disclosure. As shown in FIG. 18, a plurality of sixth feedback controls are displayed in a human-computer interaction interface 1800, each sixth feedback control being corresponding to a hearing sense, for example, including a “soft” button 1801, a “mediant” button 1802, a “treble” button 1803, and a “bass” button 1804. Then, four third test audio signals in one-to-one correspondence with “soft”, “mediant”, “treble”, and “bass” are sequentially outputted. In addition, assuming that a tap operation of the target object on the “soft” button 1801 is received during the hearing sense adjustment, “soft” may be determined as the preferred hearing sense of the target object.

In step 112, the terminal device transmits a third hearing assistance policy to the audio device.

In some embodiments, the third hearing assistance policy may be obtained by adjusting the second hearing assistance policy according to the third hearing test result. Then, the terminal device may further perform the following processing before transmitting the third hearing assistance policy to the audio device: adjusting, according to the preferred hearing sense of the target object, a gain curve included in the second hearing assistance policy. For example, assuming that the preferred hearing sense of the target object is “soft”, the gain curve included in the second hearing assistance policy may be pertinently adjusted based on a factor such as timbre corresponding to “soft”, to obtain the third hearing assistance policy.

In step 113, the audio device outputs a third audio signal adapted to the third hearing test result in place of the second audio signal.

In some embodiments, after receiving the third hearing assistance policy transmitted by the terminal device, the audio device may use the third hearing assistance policy in place of the second hearing assistance policy received in step 108. In this way, when an original audio signal is subsequently received, the received original audio signal may be adjusted by using the third hearing assistance policy, to output a third audio signal adapted to the third hearing test result (that is, an audio signal obtained through hearing sense adjustment based on the second audio signal), thereby further improving auditory experience of the user.

In the audio signal processing method provided in the embodiments of the present disclosure, a solution based on a form of a computer program is provided, which integrates functions of personalized hearing testing and configuring an audio device based on a hearing test result into the computer program. Compared with the fitting session in which the user needs to go to an offline store to configure the audio device, this lowers an operating threshold, improves efficiency in configuring the audio device, and improves auditory experience of the user.

Exemplary application of the embodiments of the present disclosure in a practical application scenario is described below by using an example that the audio device is a hearing aid. The embodiments of the present disclosure provide an autonomous fitting and machine adjusting solution based on an APP form, which integrates comprehensive functions of personalized audiometry and convenient autonomous fitting, to improve auditory experience of a hearing-impaired user in using a hearing aid.

The audio signal processing method provided in the embodiments of the present disclosure is specifically described below.

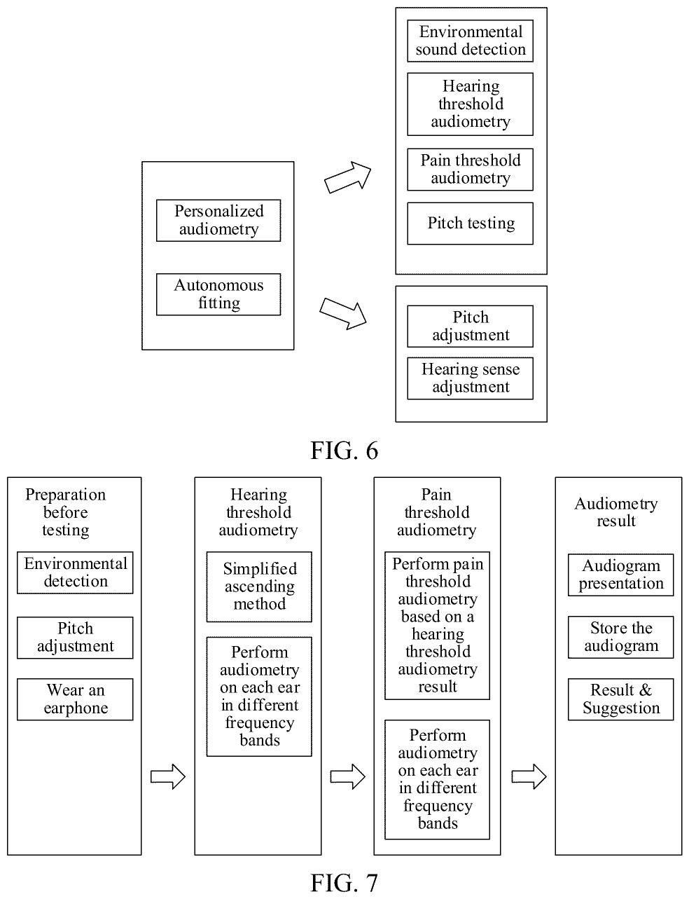

For example, FIG. 6 is a schematic diagram of functional layout according to an embodiment of the present disclosure. As shown in FIG. 6, according to functional area division, at least two buttons of “personalized audiometry” and “autonomous fitting” are included in a home page of an APP. The “personalized audiometry” includes: hearing threshold testing and pain threshold testing, that is, a user may autonomously complete hearing threshold testing and pain threshold testing through the APP. In particular, before testing, sound analysis may be performed through environmental sound detection, to determine whether an environment in which the user is currently located is quiet enough to meet an acoustic requirement of the testing. In addition, the user may autonomously complete pitch testing through the APP, to evaluate intelligibility for a speech, which is also referred to as speech clarity, that is, a percentage that the user can understand a speech signal transmitted through a specific sound transmission system. After the testing is completed, a result of the personalized audiometry may be saved as a hearing file.

The following continues to describe the “autonomous fitting” part shown in FIG. 6.

In some embodiments, after wearing a hearing aid, the user connects the hearing aid to the APP on a mobile phone through Bluetooth. Then, the user may select a hearing file. After the hearing aid is started, a parameter of the hearing aid is updated, and a basic hearing assistance function (corresponding to the first hearing assistance policy) takes effect. Next, the user may further update the parameter of the hearing aid through a pitch adjustment part designed in the APP, and a first enhanced hearing assistance function (corresponding to the second hearing assistance policy) takes effect. Subsequently, the user may further update the parameter of the hearing aid through a hearing sense adjustment part designed in the APP, and a second enhanced hearing assistance function (corresponding to the third hearing assistance policy) takes effect.

As can be seen from FIG. 6, the mobile phone APP-based autonomous fitting and machine adjusting solution provided in the embodiments of the present disclosure may be divided into two parts: personalized hearing testing and autonomous fitting. The personalized hearing testing means performing hearing tests in different frequency bands based on a terminal device (for example, a mobile phone) frequently used by a user, to obtain a personalized hearing curve graph (that is, an audiogram, also referred to as a hearing status) of the user.

For ease of expression, main constants of a quantitative description part in the embodiments of the present disclosure are as follows:

A sampling rate of a speech signal is 16000 Hz.

A frame length is 20 ms, that is, the number of samples per frame is 320 points.

If overlap-add time-frequency transform, for example, short-term Fourier transform (STFT), is performed, there is 50% overlapping. Therefore, a hop size is 320 points, and 640-point discrete Fourier transform (DFT) is performed. The hop size is the number of samples staggered between two adjacent windows.

The following first describes the personalized audiometry part.

A first part of the personalized audiometry is pure tone hearing threshold testing and pain threshold testing.

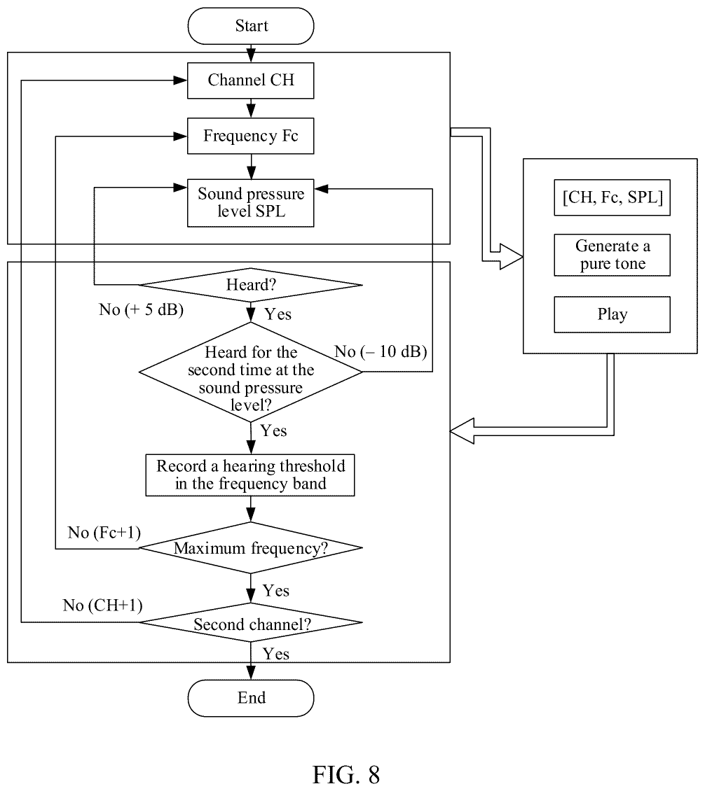

In some embodiments, FIG. 7 is a schematic flowchart of pure tone hearing threshold and pain threshold testing according to an embodiment of the present disclosure. As shown in FIG. 7, a process of pure tone hearing threshold and pain threshold testing mainly includes four steps: I. preparation before testing; II. hearing threshold audiometry; III. pain threshold audiometry; and IV. audiometry result. The following specifically describes the four steps.

### I. Preparation Before Testing

In some embodiments, the preparation before testing mainly includes environmental sound detection (for example, the “select a quiet environment” control 1002 shown in FIG. 10A), volume adjustment (for example, the “turn your mobile phone to a comfortable volume” control 1004 shown in FIG. 10A), and wearing an earphone (for example, the “put on an earphone” control 1003 shown in FIG. 10A). The preparation before testing is intended to ensure accuracy of hearing testing as much as possible. Professional-grade air conduction pure tone audiometry has strict requirements on a test environment and a test device, while a target scenario of the embodiments of the present disclosure is a conventional device and a daily environment, so that the environment requirement can be lowered to some extent by playing through the earphone. Therefore, an environmental sound detection criterion in the embodiments of the present disclosure stipulates that when an average SPL of an environment within a specific period of time (for example, 2 seconds) is less than an SPL threshold, it may be considered that a test requirement is met. For ease of description, in the embodiments of the present disclosure, the SPL threshold may be set to 40 dB, that is, if an average SPL of an environment in which the user is currently located within a specific period of time is less than 40 dB, it may be considered that a test requirement is met.

### II. Hearing Threshold Audiometry

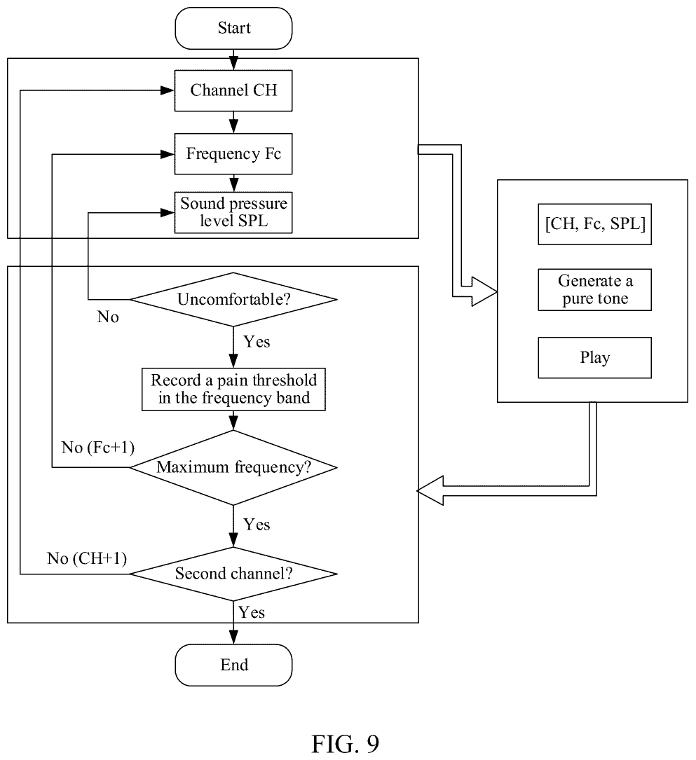

In some embodiments, FIG. 8 is a schematic flowchart of hearing threshold testing according to an embodiment of the present disclosure. As shown in FIG. 8, hearing of the user in each frequency band in an auditory frequency range may be tested by using a frequency band ascending method. For example, the auditory frequency range may be divided into six sub-bands respectively having center frequencies of 250 Hz, 500 Hz, 1000 Hz, 2000 Hz, 4000 Hz, and 8000 Hz according to response characteristics of a human ear to different frequencies. The audiometry in this embodiment of the present disclosure may be separately testing hearing of left and right ears of a subject in each frequency band by using a simplified ascending method, that is, there are a total of 12 tests. A complete ascending method requires the subject to respond five times at the same SPL, which takes a long time to test the two ears to obtain a complete audiogram. Therefore, in the audiometry method provided in this embodiment of the present disclosure, the ascending method is simplified to meet needs of general users. The simplified ascending method is to give a test sound to the subject at a preconfigured first SPL in each test, increase the test SPL by 5 dB if a tap operation of the subject on the “not heard” button 1007 shown in FIG. 10B is received, and reduce the test SPL by 10 dB if a tap operation of the subject on the “heard” button 1008 is received. This is repeated, and when a second tap operation of the subject on the “heard” button 1008 at an SPL is received, the current SPL is recorded as a hearing threshold of a current tested ear in a current frequency band, and then a next test is performed until the total of 12 tests on the two ears are completed.

### III. Pain Threshold Audiometry

In some embodiments, during pain threshold testing, to reduce a test time of the user, an initial value of the pain threshold testing may be set to a value x dB higher than the hearing threshold. For example, a value of x may be 30 dB, which may be determined according to the hearing threshold, and when the hearing threshold is higher than a threshold (for example, 60 dB), the value of x may be appropriately reduced. In addition, a protection mechanism may be added for the pain threshold testing. For example, when an SPL of a currently outputted test audio signal is higher than 75 dB, an adjustment step of the user may be forcibly reduced to prevent a sudden volume increase from damaging hearing of the user.

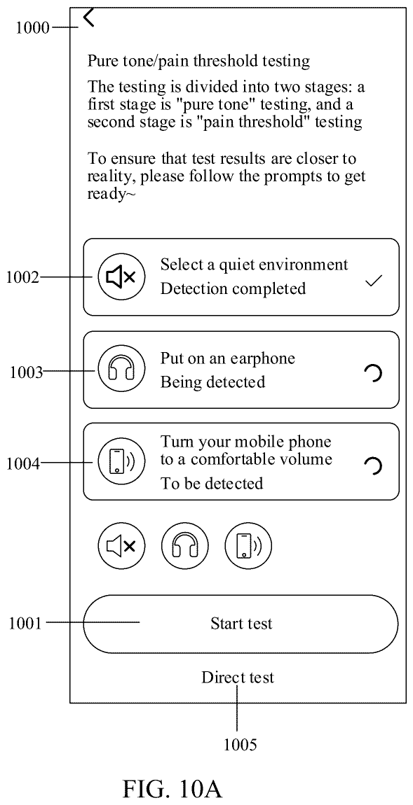

For example, FIG. 9 is a schematic flowchart of pain threshold testing according to an embodiment of the present disclosure. As shown in FIG. 9, a process of pain threshold testing includes the following steps: 1. Import an audiogram, and determine a hearing threshold in each frequency band according to the audiogram. 2. Add an increment to the hearing threshold, to obtain an initial SPL of the pain threshold testing. 3. Play a test audio signal, and update the SPL of the test audio signal according to feedback of the user. 4. Determine an SPL used when a tap operation of the user on the “ear uncomfortable” button 1014 shown in FIG. 10C is received as a pain threshold of the user in the current frequency band. 5. Perform frequency switching and determine whether tested ear switching is required. 6. Repeat steps 3, 4, and 5 until traversing all to-be-tested frequencies for the two ears. 7. Record a pain threshold testing result.

### IV. Audiometry Result

In some embodiments, after a hearing threshold and a pain threshold of the user in each sub-band in the auditory frequency range are obtained through hearing threshold testing and pain threshold testing, an audiogram of the user may be generated based on the hearing threshold and the pain threshold and saved. In addition, the audiogram may be further presented, or a related result and suggestion may be given according to the audiogram.

The following continues to describe a second part of the personalized audiometry, that is, a process of pitch testing.

For a hearing-impaired user wearing a hearing aid, a main scenario is to communicate with people. Therefore, pitch testing is mainly intended to evaluate a speech recognition rate of the user without hearing assistance means. Chinese is used as an example. Main pitches of Chinese are limited, and in the embodiments of the present disclosure, the following six pitches are used for combination: a/a, i/yi, u/wu, m/mo, s/si, and sh/shi. Certainly, an extension may be further performed on this basis, for example, h/he. For ease of description, only the six pitches are used herein. Certainly, there may be more other combinations. This is not limited in the embodiments of the present disclosure.

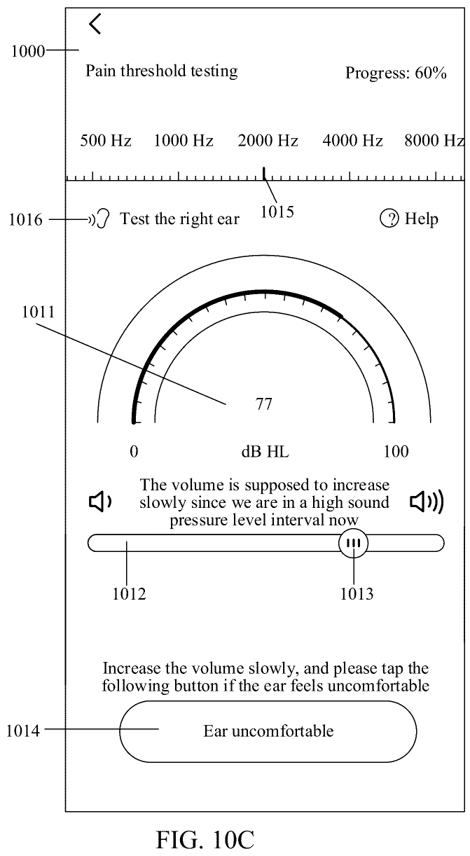

For example, FIG. 11 is a schematic flowchart of pitch testing according to an embodiment of the present disclosure. As shown in FIG. 11, main steps of pitch testing include: 1. Select a tested ear. 2. Select test sound pressure (for example, including three levels: low sound, medium sound, and loud sound). 3. Generate and play test audio signals of the six pitches. 4. Record feedback of the user, and determine whether the feedback is correct or incorrect. For example, the APP plays a pitch in background, assumed to be a/a, and the user may perform selection according to what the user hears. For example, assuming that a tap operation of the user on the “a/a” button 1202 shown in FIG. 12 is received, it is recorded in background that the user hears the pitch correctly. If a tap operation of the user on another button or the “inaudible” button 1208 shown in FIG. 12 is received, it is recorded in background that the user hears the pitch incorrectly. 5. If the user distinguishes the pitch incorrectly, play the pitch for the second time and record a feedback status. 6. Repeat the foregoing steps until traversing the two ears and the six pitches.

The following describes a process of “autonomous fitting”.

A first part of the autonomous fitting is to obtain basic hearing for the hearing-impaired user through the hearing aid according to the audiogram obtained through the personalized audiometry.

In some embodiments, a solution of the basic hearing assistance function is calculating a gain of each frequency band according to a personalized hearing status of the user (for example, by loading the audiogram), and then compensating for hearing loss of the user in a fullband range by using different gains of frequency bands, to improve a perception capability of the user in a hearing loss band, thereby improving speech intelligibility of the user and meeting a need of daily communication. The following specifically describes the solution of the basic hearing assistance function.

First, the audiogram (that is, a hearing test result obtained through personalized hearing testing) of the user is loaded. As described above, the audiogram may be obtained by the user through a personalized audiometry module in the APP provided in the embodiments of the present disclosure, or may be obtained from a third party through direct input (for example, an accurate pure tone audiogram is obtained from a professional organization). This is not limited in the embodiments of the present disclosure.

Then, a gain value of each frequency band is calculated according to the audiogram and a prescription formula. Herein, the prescription formula is a formula for determining the gain value of each frequency band according to a hearing threshold in the frequency band. A prescription formula given in Table 1 is used as an example. TH represents a corresponding hearing threshold, and G represents a calculated gain. The formula in Table 1 is a non-linear prescription formula, and the gain value of each frequency band may be calculated according to an SPL of an input audio signal and the hearing threshold. A specific gain calculation process is as follows: First, the SPL of the input audio signal is calculated. Then, sound intensity is determined according to the SPL of the input audio signal, to determine a gain interval of the formula in Table 1. The SPL being below 40 dB indicates a low-intensity sound, the SPL being 40 dB to 65 dB indicates a comfort-zone sound, and the SPL being 65 dB to 90 dB indicates a high-intensity sound. Subsequently, the hearing threshold in each frequency band is determined, and is substituted into the prescription formula given in Table 1 for calculation, to obtain a gain value of a current frequency band.

In the embodiments of the present disclosure, a gain outputted by the prescription formula is limited, that is, to ensure that strength of an equalized audio signal does not cause further loss to hearing of the user, when a sum of the SPL of the input audio signal and the gain value exceeds a pain threshold of the user, a part of the gain exceeding the pain threshold is removed.

TABLE 1
Prescription formula

| | |
|---|---|
| Low-intensity | (1) 0-20 dB HL: G = 0 |
| sound gain | (2) 20-60 dB HL: G = TH − 20 |
| | (3) TH ≥60 dB HL: G = TH − 20 − 0.5 × (TH − 60) |
| Comfort-zone | (1) 0-20 dB HL: G = 0 |
| sound gain | (2) 20-60 dB HL: G = 0.6 × (TH − 20) |
| | (3) TH ≥60 dB HL: G = 0.8 × (TH − 23) |
| High-intensity | (1) 0-40 dB HL: G = 0 |
| sound gain | (2) TH ≥40 dB HL: G = 0.1 × (TH − 40)^{1.4} |

Further, gain values respectively corresponding to the six sub-bands are substituted into a filter function for calculation, to obtain the filter bank parameter. The filter bank may include a shelving filter and a peak filter. The shelving filter may include a low shelf filter and a high shelf filter. The low shelf filter is characterized in that a high-frequency part is straight-through and a low-frequency part is adjustable (that is, it may be configured to adjust a gain of a low-frequency sub-band. The high shelf filter is characterized in that a low-frequency part is straight-through and a high-frequency part is adjustable (that is, it may be configured to adjust a gain of a high-frequency sub-band. The peak filter is located between the low shelf filter and the high shelf filter, and configured to raise a center frequency response, and adjust a gain of an intermediate sub-band.

Further, in the embodiments of the present disclosure, “backward” filter parameter calculation is performed, that is, a filter parameter corresponding to a high-frequency sub-band is first determined, and then a filter parameter of a low-frequency sub-band is calculated according to a characteristic of a frequency response that undergoes filtering, to obtain filter parameters level by level.

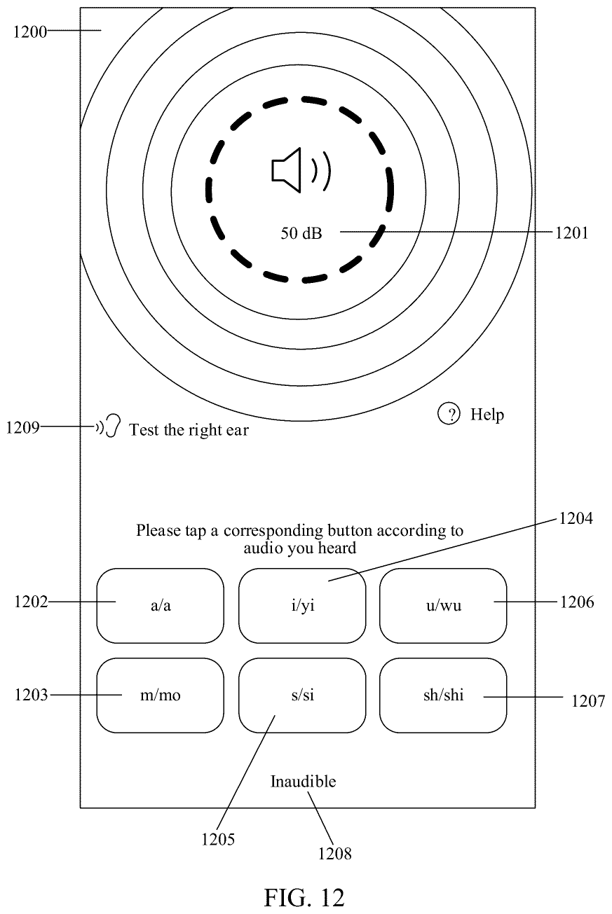

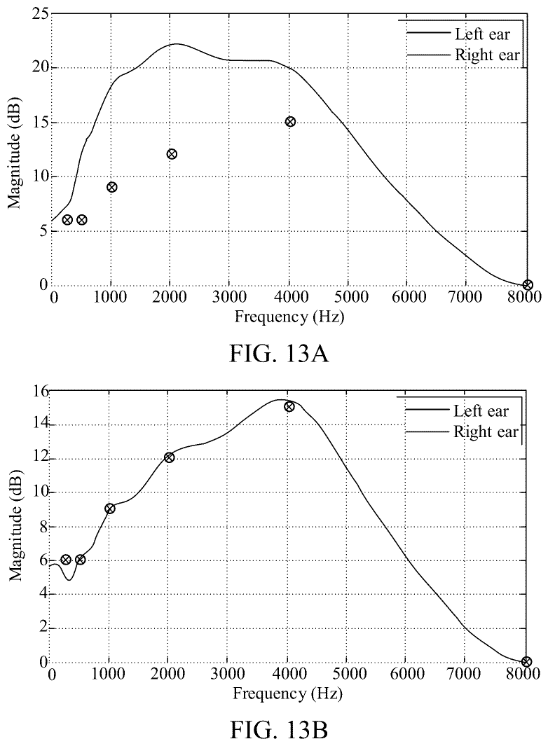

For example, FIG. 13A is a schematic diagram of a frequency response curve corresponding to an offline fitting session, and FIG. 13B is a schematic diagram of a frequency response curve according to an embodiment of the present disclosure. Circles and x in the figures represent desired gains of corresponding sub-bands, respectively. As can be seen with reference to FIG. 13A and FIG. 13B, the solution provided in this embodiment of the present disclosure can be closer to a desired frequency response curve than a method of directly calculating a single filter parameter. For example, in the solution provided in this embodiment of the present disclosure, a gain at a target sub-band is closer to a desired value.

Finally, the input audio signal is filtered for equalization to obtain an output audio signal (corresponding to the first audio signal). The input original audio signal is sequentially filtered by filters (from a low frequency to a high frequency), and the output audio signal obtained is an equalized audio signal.

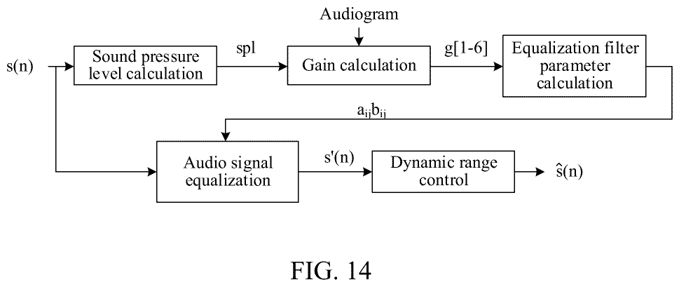

For example, FIG. 14 is a schematic flowchart of personalized equalization according to an embodiment of the present disclosure. As shown in FIG. 14, a time domain signal s(n) obtained by splicing an n^{th} frame and an (n−1)^{th} frame is inputted into an SPL calculation module to obtain an SPL of the current frame of signal. An SPL calculation formula is as follows:

$$spl = 10 {log}_{10} \frac{\sum {s ( n )}^{2}}{{( 2 \times 1 {0}^{- 5} )}^{2}}$$

For example, it is assumed that the calculated SPL is spl=60 dB, and hearing thresholds corresponding to the left ear in the six sub-bands are pta_L=[30 35 35 40 45 45]. First, a corresponding gain interval is determined according to the SPL. Refer to Table 1. The input SPL of 60 dB corresponds to the comfort-zone sound. Then, a corresponding gain may be calculated according to the hearing threshold in each sub-band. Herein, the first sub-band is used as an example. The hearing threshold is 30 dB, corresponding to a formula (2) of the comfort-zone sound interval, and is substituted to calculate g[1]=0.6×(30−20)=6 dB. Similarly, gain values of the other five sub-bands may be calculated sequentially.

Subsequently, when filter parameters are calculated based on the gain values g, in this embodiment of the present disclosure, a “backward” calculation method is used to get closer to a desired response curve. That is, a parameter of a high shelf filter is first calculated according to a gain value g[6] of the sixth sub-band, and then a filter parameter of the fifth sub-band is calculated according to a difference g′[5] between a gain value g[5] of the fifth sub-band and a frequency response h_6[5] of the filter at the fifth sub-band, and so on, to obtain a parameter aᵢⱼbᵢⱼ of the entire filter bank. Details are not described herein in this embodiment of the present disclosure.

After the filter bank parameter is obtained, the input original audio signal may be sequentially processed by six filters from a low frequency to a high frequency, to obtain an audio signal s′(n) that undergoes personalized equalization. The filtering operation is embodied as convolution in time domain and corresponding frequency bin multiplication in frequency domain, which are basic operations of signal processing. Details are not described herein in this embodiment of the present disclosure.

Finally, to prevent a “clipping” phenomenon from occurring in the output audio signal and affecting a hearing sense, in this embodiment of the present disclosure, a DRC module is further added after output equalization, to protect integrity of the audio signal.

The following continues to describe a second part of the autonomous fitting, that is, a process of pitch adjustment.

The pitch adjustment is intended to use, on the basis of implementing the basic hearing assistance function, the pitch adjustment part designed in the APP, that is, perform pitch testing in real time after the hearing-impaired user wears the hearing aid, to finely adjust a parameter according to a testing result, and update an adjusted parameter to the hearing aid by using the Bluetooth protocol, so as to improve the hearing sense of the user (that is, implement the first enhanced hearing assistance function).

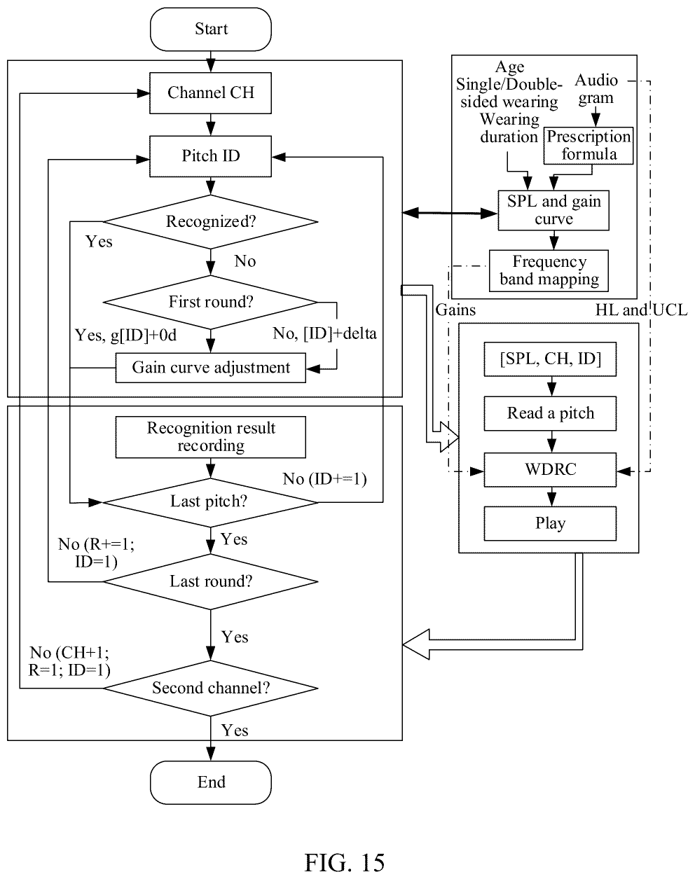

In some embodiments, FIG. 15 is a schematic flowchart of pitch adjustment according to an embodiment of the present disclosure. As shown in FIG. 15, a main procedure of pitch adjustment is as follows: 1. Determine a gain factor (a factor 1, where the gain factor is aimed at an overall gain of an audio signal) according to an age, a wearing side, and wearing years of the user. 2. Import an audiogram. The audiogram includes hearing thresholds and pain thresholds of the user in different frequency bands. 3. Calculate three gain curves according to the gain factor, the hearing thresholds, and the pain thresholds by using a prescription formula, which respectively correspond to low sound, medium sound, and loud sound. 4. Perform interpolation through frequency band mapping so that the number of sub-bands of the gain curve is consistent with the number of channels of a personalized equalization filter (corresponding to the filter bank). The frequency band mapping may be implemented through linear interpolation. 5. Perform personalized hearing assistance processing (for example, amplification) on a signal with a given pitch according to the gain curve, play the signal to the user for audiometry, and record a recognition result of the user. 6. Pertinently compensate the gain curve for a pitch incorrectly recognized by the user. Different error situations correspond to different compensation, while an adjustment amount of the compensation may be preset without manual adjustment of the user. 7. Repeat steps 5 and 6 until the adjustment is completed. 8. Store a current adjustment result (corresponding to the second hearing assistance policy).

The pitch adjustment is intended to play any pitch and adjust perception of the user on the pitch at different frequencies according to feedback of the user, which can help improve a speech recognition rate. In addition, an interaction mode of pitch adjustment provided in the embodiments of the present disclosure is also different from that of fitting provided in other fitting solutions. In the other fitting solutions, autonomous adjustment is generally performed in three stages, which does not have an enough channel resolution, but has a high professional requirement, leading to a high operating threshold. In contrast, as shown in FIG. 16, in the solution provided in the embodiments of the present disclosure, the user only needs to feed back a recognition status through a button, and compensation is adaptively performed in background according to recognition results of the user on different pitches. For the user, an operating threshold is low and user experience is good. A latest parameter selected by the user may be updated to the hearing aid by using the Bluetooth protocol, so that the user can obtain a better pitch hearing effect.

The following describes a third part of the autonomic fitting, that is, a process of hearing sense adjustment.

A main process of hearing sense adjustment is using, on the basis of implementing the basic hearing assistance function and the first enhanced hearing assistance function, the hearing sense adjustment part designed in the APP, to perform hearing sense testing in real time after the hearing-impaired user wears the hearing aid, to finely adjust a parameter according to a hearing sense testing result, and update an adjusted parameter to the hearing aid by using the Bluetooth protocol, so as to improve a hearing sense of the user (that is, implement the second enhanced hearing assistance function).

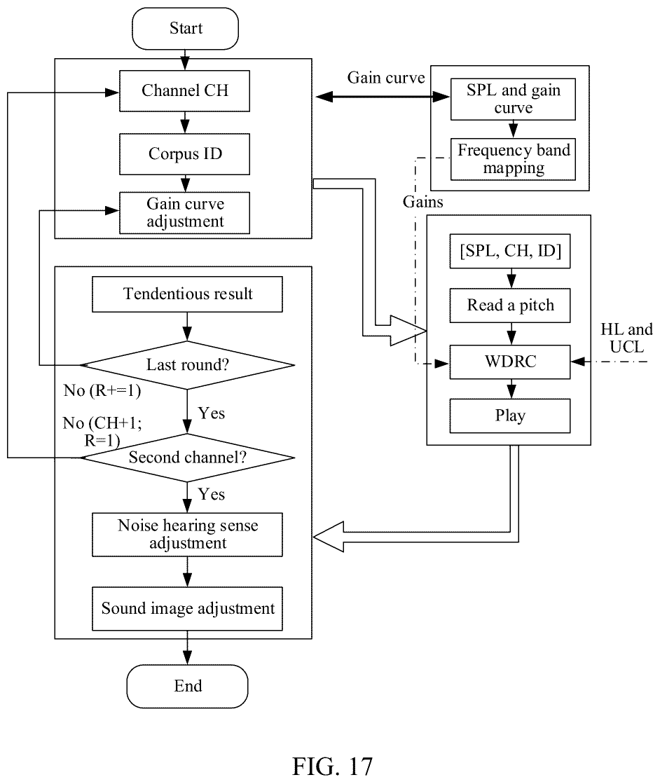

For example, FIG. 17 is a schematic flowchart of hearing sense adjustment according to an embodiment of the present disclosure. As shown in FIG. 17, a main procedure of hearing sense adjustment is as follows: 1. Import a hearing assistance solution that undergoes pitch adjustment. 2. Select any speech signal in a speech database, generate four types of candidate speech signals according to the hearing assistance solution, and play the signals to the user. The four types of candidate speech signals are an original hearing assistance solution, higher, lower, and a clearer speech, respectively. 3. Estimate a tendency of the user according to selection performed by the user, and further strengthen the tendency. For example, if the user selects B, as B corresponds to a low characteristic, the low characteristic is further strengthened in a next round of adjustment. In this case, an adjustment amount may be preset according to an algorithm without manual adjustment of the user. 4. After a total of N rounds of adjustment, consider that pure speech hearing sense adjustment is completed, and store the hearing assistance solution. 5. Perform hearing sense adjustment for a noisy speech. For example, noise (which may be several types of common sounds that cause discomfort to the user, such as a flute sound and a machine sound) at different levels (as close as possible to true values) may be used together with a speech signal for hearing assistance processing. 6. Record a speech hearing sense and whether the noise causes discomfort that are fed back by the user, and adjust a gain curve according to a feedback result of the user. 7. Perform sound image correction, that is, play a signal to two ears at the same time, and adjust gains of the two ears according to a perceived sound image position fed back by the user, so that a sound image is located right in the middle to implement binaural equalization. 8. Store the hearing assistance solution (corresponding to the third hearing assistance policy).

For example, FIG. 18 is a schematic diagram of an application scenario of an audio signal processing method according to an embodiment of the present disclosure. As shown in FIG. 18, the user may select four candidate policies, sounds adjusted by using corresponding policies are played in background, and the user taps next after selection according to the user's own preference. A latest parameter selected by the user may be updated to the hearing aid by using the Bluetooth protocol, to obtain a better pitch hearing effect.

The following describes, by comparing the other fitting schemes with the audio signal processing method provided in the embodiments of the present disclosure, technical effects brought by the embodiments of the present disclosure.

In other fitting schemes, first, hearing aids as professional equipment usually need face-to-face communication with audiologists in offline stores for fitting, which leads to poor timeliness. In addition, in the other fitting schemes, hearing is generally assisted based on an audiometry result by using a general prescription formula. However, considering uniqueness of each person's hearing, it is necessary to implement more personalized hearing assistance based on user feedback. In addition, in the aspect of autonomous fitting, another fitting solution is to perform adjustment by stages, and directly provide a gain adjustment interface of each frequency band to a user for the user to perform adjustment. However, this adjustment manner has a high professional requirement and a high operating threshold. In addition, when the user cannot control an adjustment amount well, a hearing assistance effect is reduced instead.

Embodiments of the present disclosure provide fitting solutions with the following improvement directions:
1. Support the user in performing autonomous testing at any time to learn a current hearing status. In addition, for a common user (for example, a user who has healthy hearing or has not been clinically defined as hearing lost), it is also beneficial to hearing health of the common user to detect a hearing status at any time based on a lightweight APP.
2. Support pertinent hearing assistance based on a personalized hearing status of the user. For example, it is better to assist hearing according to a frequency of hearing loss. Optimizing a hearing assistance effect of a hearing aid through autonomous fitting can better meet a personalized requirement. An interaction mode of machine adjustment is optimized, so that machine adjustment is more convenient and more efficient.

In view of this, in the embodiments of the present disclosure, comprehensive functions of personalized audiometry and convenient autonomous fitting are integrated into an APP, so that a user can test hearing only by interacting with the APP, thereby meeting a requirement of the user for hearing testing at any time. In addition, during configuration on a hearing aid, in the embodiments of the present disclosure, a corresponding hearing assistance policy is generated based on a personalized hearing test result of the user, so that the generated hearing assistance policy can better meet a personalized requirement of the user. Further, in the solution provided in the embodiments of the present disclosure, during pitch adjustment, compensation may be performed pertinently according to a feedback result of the user, where the user only needs to feed back a recognition status through a button, thereby lowering an operating threshold for the user, making the adjustment process more convenient and quick, and improving user experience.

The following continues to describe an exemplary structure in which the audio signal processing apparatus 255 provided in the embodiments of the present disclosure is implemented as software modules. In some embodiments, as shown in FIG. 2A, the software modules stored in the audio signal processing apparatus 255 of the memory 250 may include: a display module 2551, an output module 2552, and a transmitting module 2553.

The display module 2551 is configured to display a hearing test control in a human-computer interaction interface. The output module 2552 is configured to output a first test audio signal in response to a trigger operation on the hearing test control. The display module 2551 is further configured to display a first hearing test result of a target object in response to a feedback operation on the first test audio signal. The transmitting module 2553 is configured to transmit, to an audio device in response to a configuration operation on the audio device, a first hearing assistance policy generated according to the first hearing test result, where the first hearing assistance policy is configured to be applied to the audio device to output a first audio signal adapted to the first hearing test result.

In some embodiments, the first hearing test result includes at least one of a hearing parameter and a speech recognition capability parameter, and the first test audio signal includes at least one of the following types of test audio signals: a hearing test audio signal for testing hearing of the target object, and a speech recognition capability test audio signal for testing a speech recognition capability of the target object. The audio signal processing apparatus 255 further includes a generation module 2554, configured to generate a hearing parameter of the target object in response to a feedback operation on the hearing test audio signal; and generate a speech recognition capability parameter of the target object in response to a feedback operation on the speech recognition capability test audio signal. The display module 2551 is further configured to display a hearing test result of the target object, the hearing test result including at least one of the hearing parameter and the speech recognition capability parameter.

In some embodiments, the hearing parameter includes a hearing threshold of the target object in each sub-band in an auditory frequency range. The generation module 2554 is further configured to perform the following processing for any sub-band in the auditory frequency range: displaying a first feedback control and a second feedback control in the human-computer interaction interface, the first feedback control being configured to indicate that the hearing test audio signal is not heard, and the second feedback control being configured to indicate that the hearing test audio signal is heard; re-outputting the hearing test audio signal in a manner higher than an SPL of current output in response to a trigger operation on the first feedback control; re-outputting the hearing test audio signal in a manner lower than an SPL of current output in response to a trigger operation on the second feedback control; and for any SPL used for current output, determining the any SPL as a hearing threshold of the target object in the sub-band in response to receiving a second trigger operation on the second feedback control at the any SPL.

In some embodiments, the display module 2551 is further configured to perform the following processing during the displaying of the first feedback control and the second feedback control in the human-computer interaction interface: displaying an SPL control in the human-computer interaction interface, the SPL control being configured to indicate an SPL of the currently outputted hearing test audio signal.

In some embodiments, the hearing parameter includes a pain threshold of the target object in each sub-band in an auditory frequency range. The generation module 2554 is further configured to perform the following processing for any sub-band in the auditory frequency range: displaying a first adjustment control and a third feedback control in the human-computer interaction interface, the first adjustment control being used for adjusting an SPL, and the third feedback control being configured to indicate that physiological discomfort occurs in hearing the hearing test audio signal; and in response to a trigger operation on the third feedback control, determining an SPL in response to that the trigger operation is received as a pain threshold of the target object in the sub-band.

In some embodiments, the display module 2551 is further configured to display a plurality of fourth feedback controls in the human-computer interaction interface, each fourth feedback control being corresponding to a pitch. The output module 2552 is further configured to sequentially output a plurality of speech recognition capability test audio signals. The audio signal processing apparatus 255 further includes a recording module 2555, configured to record a fourth feedback control triggered in the plurality of fourth feedback controls each time the speech recognition capability test audio signal is outputted. The generation module 2554 is further configured to generate the speech recognition capability parameter of the target object based on pitches respectively corresponding to the plurality of speech recognition capability test audio signals and fourth feedback controls respectively triggered in a plurality of test processes.

In some embodiments, the display module 2551 is further configured to perform the following processing during the displaying of the plurality of fourth feedback controls in the human-computer interaction interface: displaying a decibel control in the human-computer interaction interface, the decibel control being configured to indicate a decibel value of the currently outputted speech recognition capability test audio signal.

In some embodiments, the audio signal processing apparatus 255 further includes a detection module 2556 and a turn module 2557. The detection module 2556 is configured to: before the first test audio signal is outputted, detect an SPL of an environment in which the target object is located. The turn module 2557 is configured to turn to the operation of outputting the first test audio signal in response to that an average SPL of the environment within a set duration is less than an SPL threshold.

In some embodiments, the audio signal processing apparatus 255 further includes a determining module 2558 and a combination module 2559. The determining module 2558 is configured to: before the first hearing assistance policy generated according to the first hearing test result is transmitted to the audio device, determine, in descending order of frequencies of each sub-band in an auditory frequency range, a filter parameter of each sub-band based on the first hearing test result, a filter parameter of a low-frequency sub-band being determined based on a filter parameter of a high-frequency sub-band. The combination module 2559 is configured to perform combination on the filter parameter of each sub-band, and use a combined filter bank parameter as the first hearing assistance policy for the target object.

In some embodiments, the first hearing test result includes a hearing threshold of the target object in each sub-band. The determining module 2558 is further configured to obtain a gain value of each sub-band based on the hearing threshold of the target object in each sub-band and a prescription formula; and obtain, in descending order of frequencies, the filter parameter of each sub-band based on the gain value of each sub-band.

In some embodiments, the auditory frequency range includes N sub-bands, N being an integer greater than 1. The determining module 2558 is further configured to substitute a gain value of the N^{th} sub-band into a filter function for calculation to obtain a filter parameter of the Ni sub-band; and determine a filter parameter of an it sub-band based on a difference between a gain value of the it sub-band and a frequency response of a filter of an (i+1)^{th} sub-band at the it sub-band, a value range of i meeting 1≤i≤N−1, and a frequency of the (i+1)^{th} sub-band being greater than a frequency of the it sub-band.

In some embodiments, the determining module 2558 is further configured to amplify the first audio signal according to at least one gain curve, to obtain a second test audio signal of at least one volume. The generation module 2554 is further configured to generate a second hearing test result of the target object in response to a feedback operation on the second test audio signal. The transmitting module 2553 is further configured to transmit a second hearing assistance policy to the audio device, the second hearing assistance policy being obtained by adjusting the first hearing assistance policy according to the second hearing test result, and being applied to the audio device to output a second audio signal adapted to the second hearing test result in place of the first audio signal.

In some embodiments, the display module 2551 is further configured to display a second adjustment control and a plurality of fifth feedback controls in the human-computer interaction interface, the second adjustment control being used for adjusting a gain of the second test audio signal, and each fifth feedback control being corresponding to a pitch. The output module 2552 is further configured to sequentially output a plurality of second test audio signals. The recording module 2555 further configured to record a fifth feedback control triggered in the plurality of fifth feedback controls each time the second test audio signal is outputted. The generation module 2554 is further configured to generate the second hearing test result of the target object based on pitches respectively corresponding to the plurality of the second test audio signals and fifth feedback controls respectively triggered in a plurality of test processes.

In some embodiments, the display module 2551 is further configured to perform the following processing during the displaying of the second adjustment control and the plurality of fifth feedback controls in the human-computer interaction interface: displaying a plurality of volume controls in the human-computer interaction interface, a volume represented by a volume control in a selected state being used as a volume used during outputting of the second test audio signal.

In some embodiments, the second hearing test result includes a pitch incorrectly recognized by the target object. The audio signal processing apparatus 255 further includes a compensation module 25510, configured to: before the second hearing assistance policy is transmitted to the audio device, pertinently compensate the first hearing assistance policy according to the pitch incorrectly recognized by the target object, to obtain the second hearing assistance policy.

In some embodiments, the determining module 2558 is further configured to determine a gain factor of the first audio signal according to characteristic information of the target object before the first audio signal is amplified according to the at least one gain curve. The generation module 2554 is further configured to generate the at least one gain curve according to the hearing parameter included in the first hearing test result, the gain factor, and the prescription formula, each gain curve being corresponding to a volume, and the hearing parameter including at least one of the hearing threshold and the pain threshold of the target object in each sub-band in the auditory frequency range. The audio signal processing apparatus 255 further includes an interpolation module 25511, configured to perform interpolation processing on each gain curve through frequency band mapping, so that the number of sub-bands of the gain curve is consistent with the number of channels of a filter bank.

In some embodiments, the audio signal processing apparatus 255 further includes an adjustment module 25512, configured to respectively adjust the second audio signal based on a plurality of candidate hearing sense adjustment policies, to correspondingly obtain a plurality of third test audio signals. The generation module 2554 is further configured to generate a third hearing test result of the target object in response to a feedback operation on the plurality of third test audio signals. The transmitting module 2553 is further configured to transmit a third hearing assistance policy to the audio device, the third hearing assistance policy being obtained by adjusting the second hearing assistance policy according to the third hearing test result, and being applied to the audio device to output a third audio signal adapted to the third hearing test result in place of the second audio signal.

In some embodiments, the third hearing test result includes a preferred hearing sense of the target object. The display module 2551 is further configured to display a plurality of sixth feedback controls in the human-computer interaction interface, each sixth feedback control being corresponding to a hearing sense. The output module 2552 is further configured to sequentially output a plurality of third test audio signals in one-to-one correspondence with the plurality of sixth feedback controls. The determining module 2558 is further configured to determine a hearing sense corresponding to a sixth feedback control triggered in the plurality of sixth feedback controls as the preferred hearing sense of the target object.

In some embodiments, the adjustment module 25512 is further configured to: before the third hearing assistance policy is transmitted to the audio device, adjust, according to the preferred hearing sense of the target object, a gain curve included in the second hearing assistance policy, to obtain the third hearing assistance policy.

In some embodiments, the display module 2551 is further configured to display a historical hearing test result of the target object in the human-computer interaction interface in response to existence of the historical hearing test result and the historical hearing test result being within a validity period. The transmitting module 2553 is further configured to transmit, to the audio device in response to a configuration operation on the audio device, a fourth hearing assistance policy generated according to the historical hearing test result, the fourth hearing assistance policy being applied to the audio device to output a fourth audio signal adapted to the historical hearing test result.

In some other embodiments, as shown in FIG. 2A, the software modules stored in the audio signal processing apparatus 255 of the memory 250 may include: an obtaining module 25513, a determining module 2558, a combination module 2559, and a transmitting module 2553. The obtaining module 25513 is configured to obtain a first hearing test result of a target object. The determining module 2558 is configured to determine, in descending order of frequencies of each sub-band in an auditory frequency range, a filter parameter of each sub-band based on the first hearing test result, a filter parameter of a low-frequency sub-band being determined based on a filter parameter of a high-frequency sub-band. The combination module 2559 is configured to perform combination on the filter parameter of each sub-band, and use a combined filter bank parameter as a first hearing assistance policy for the target object. The transmitting module 2553 is configured to transmit the first hearing assistance policy to an audio device, where the first hearing assistance policy is configured to be applied to the audio device to output a first audio signal adapted to the first hearing test result.

The following continues to describe an exemplary structure in which the audio signal processing apparatus 355 provided in the embodiments of the present disclosure is implemented as software modules. In some embodiments, as shown in FIG. 2B, the software modules stored in the audio signal processing apparatus 355 of the memory 350 may include: a receiving module 3551 and an output module 3552.

The receiving module 3551 is configured to receive a first hearing assistance policy for a target object, the first hearing assistance policy including a filter bank parameter, the filter bank parameter including a filter parameter of each sub-band in an auditory frequency range, the filter parameter of each sub-band being determined in descending order of frequencies based on a first hearing test result of the target object, and a filter parameter of a low-frequency sub-band being determined based on a filter parameter of a high-frequency sub-band. The output module 3552 is configured to output, according to the first hearing assistance policy, a first audio signal adapted to the first hearing test result.

In some embodiments, the receiving module 3551 is further configured to receive a second hearing assistance policy for the target object, the second hearing assistance policy being obtained by adjusting the first hearing assistance policy according to a second hearing test result, the second hearing test result being obtained based on a feedback operation of the target object on a second test audio signal, and the second test audio signal being obtained by amplifying the first audio signal according to a gain curve. The output module 3552 is further configured to output, according to the second hearing assistance policy, a second audio signal adapted to the second hearing test result in place of the first audio signal.

In some embodiments, the receiving module 3551 is further configured to receive a third hearing assistance policy for the target object, the third hearing assistance policy being obtained by adjusting the second hearing assistance policy according to a third hearing test result, the third hearing test result being obtained based on a feedback operation of the target object on a plurality of third test audio signals, and the plurality of third test audio signals being obtained by adjusting the second audio signal based on a plurality of candidate hearing sense adjustment policies. The output module 3552 is further configured to output, according to the third hearing assistance policy, a third audio signal adapted to the third hearing test result in place of the second audio signal.

In some embodiments, the output module 3552 is further configured to control, in ascending order of frequencies, a filter of each sub-band in a filter bank to sequentially filter an original audio signal according to a filter parameter of the corresponding sub-band in the filter bank parameter, to obtain the first audio signal adapted to the first hearing test result.

The description of the apparatus in the embodiments of the present disclosure is similar to the description of the method embodiment, and the apparatus has beneficial effects similar to those of the method embodiment, and therefore details are not described again. Technical details that are not explained in detail in the audio signal processing apparatus provided in the embodiments of the present disclosure can be understood with reference to the description of any one of FIG. 3 to FIG. 5.

An embodiment of the present disclosure provides a computer program product or a computer program, the computer program product or the computer program including a computer instruction, the computer instruction being stored in a computer-readable storage medium, A processor of a computer device reads the computer instruction from the computer-readable storage medium, and the processor executes the computer instruction, to cause the computer device to execute the audio signal processing method in the embodiments of the present disclosure.

An embodiment of the present disclosure provides a computer-readable storage medium storing an executable instruction, when executed by a processor, causing the processor to execute the audio signal processing method provided in the embodiments of the present disclosure, for example, the audio signal processing method shown in any one of FIG. 3 to FIG. 5.

In some embodiments, the computer-readable storage medium may be an FRAM, a ROM, a PROM, an EPROM, an EEPROM, a flash memory, a magnetic surface storage, an optical disc, a CD-ROM, or other memories; or may be various devices including one or any combination of the memories.

In some embodiments, the executable instruction may be in a form of a program, software, a software module, a script, or code, written in any form of programming language (including compiling or interpreting languages, or declarative or procedural languages), and may be deployed in any form, including being deployed as a stand-alone program, a module, a component, a subroutine, or other units suitable for use in a computing environment.

In an example, the executable instruction may but not necessarily correspond to a file in a file system, and may be stored as a part of a file storing other programs or data, for example, stored in one or more scripts in a hypertext markup language (HTML) file, stored in a single file specific to a program discussed, or stored in a plurality of collaborative files (for example, a file storing one or more modules, subroutines, or portions of code).

In an example, the executable instruction may be deployed on one electronic device, a plurality of electronic devices at one location, or a plurality of electronic devices distributed at a plurality of locations and interconnected through a communication network for execution.

The foregoing descriptions are merely exemplary embodiments of the present disclosure and are not intended to limit the protection scope of the present disclosure. Any modification, equivalent replacement, improvement, and the like made without departing from the spirit and range of the present disclosure shall fall within the protection scope of the present disclosure.

What is claimed is:

1. An audio signal processing method, applied to an electronic device, the method comprising:
displaying a hearing test control in a human-computer interaction interface;
outputting a first test audio signal in response to a trigger operation on the hearing test control;
displaying a first hearing test result of a target object in response to a feedback operation on the first test audio signal; and
transmitting, to an audio device in response to a configuration operation on the audio device, a first hearing assistance policy generated according to the first hearing test result, wherein the first hearing assistance policy is configured to be applied to the audio device to output a first audio signal adapted to the first hearing test result.

2. The method according to claim 1, wherein
the first hearing test result comprises at least one of a hearing parameter and a speech recognition capability parameter, and the first test audio signal comprises at least one of: a hearing test audio signal for testing hearing of the target object, and a speech recognition capability test audio signal for testing a speech recognition capability of the target object; and
the displaying a first hearing test result of a target object in response to a feedback operation on the first test audio signal comprises:
generating a hearing parameter of the target object in response to a feedback operation on the hearing test audio signal;
generating a speech recognition capability parameter of the target object in response to a feedback operation on the speech recognition capability test audio signal; and
displaying a hearing test result of the target object, the hearing test result comprising at least one of the hearing parameter and the speech recognition capability parameter.

3. The method according to claim 2, wherein
the hearing parameter comprises hearing thresholds of the target object in a plurality of sub-bands in an auditory frequency range; and
the generating a hearing parameter of the target object in response to a feedback operation on the hearing test audio signal comprises: for one sub-band of the plurality of sub-bands in the auditory frequency range:
displaying a first feedback control and a second feedback control in the human-computer interaction interface, the first feedback control indicating that the hearing test audio signal is not heard by the target object, and the second feedback control indicating that the hearing test audio signal is heard by the target object;
re-outputting the hearing test audio signal in a manner higher than a sound pressure level of a current output in response to a trigger operation on the first feedback control;
re-outputting the hearing test audio signal in a manner lower than a sound pressure level of the current output in response to a trigger operation on the second feedback control; and
for a sound pressure level corresponding to the current output, determining the sound pressure level as a hearing threshold of the target object in the sub-band in response to receiving the trigger operation on the second feedback control at the sound pressure level twice.

4. The method according to claim 3, wherein during the displaying a first feedback control and a second feedback control in the human-computer interaction interface, the method further comprises:
displaying a sound pressure level control in the human-computer interaction interface, the sound pressure level control being configured to indicate a sound pressure level of the currently outputted hearing test audio signal.

5. The method according to claim 2, wherein
the hearing parameter comprises pain thresholds of the target object in a plurality of sub-bands in an auditory frequency range; and
the generating a hearing parameter of the target object in response to a feedback operation on the hearing test audio signal comprises: for one sub-band of the plurality of sub-bands in the auditory frequency range:
displaying a first adjustment control and a third feedback control in the human-computer interaction interface, the third feedback control being configured to indicate that physiological discomfort occurs on the target object in hearing the hearing test audio signal;
adjusting a sound pressure level of the currently outputted hearing test audio signal in response to a trigger operation on the first adjustment control; and
in response to a trigger operation on the third feedback control, determining a sound pressure level in response to that the trigger operation is received as a pain threshold of the target object in the sub-band.

6. The method according to claim 2, wherein the generating a speech recognition capability parameter of the target object in response to a feedback operation on the speech recognition capability test audio signal comprises:
displaying a plurality of fourth feedback controls in the human-computer interaction interface, each fourth feedback control being corresponding to a pitch;
sequentially outputting a plurality of speech recognition capability test audio signals, and recording a fourth feedback control triggered in the plurality of fourth feedback controls each time the speech recognition capability test audio signal is outputted; and
generating the speech recognition capability parameter of the target object based on pitches respectively corresponding to the plurality of speech recognition capability test audio signals and fourth feedback controls respectively triggered in a plurality of test processes.

7. The method according to claim 6, wherein during the displaying a plurality of fourth feedback controls in the human-computer interaction interface, the method further comprises:
displaying a decibel control in the human-computer interaction interface, the decibel control being configured to indicate a decibel value of the currently outputted speech recognition capability test audio signal.

8. The method according to claim 1, wherein the method further comprises:
detecting a sound pressure level of an environment in which the target object is located; and
turning to the operation of outputting a first test audio signal in a case that an average sound pressure level of the environment within a set duration is less than a sound pressure level threshold.

9. The method according to claim 1, wherein the method further comprises:
determining, in descending order of frequencies of a plurality of sub-bands in an auditory frequency range, a filter parameter of each sub-band based on the first hearing test result, a filter parameter of a low-frequency sub-band being determined based on a filter parameter of a high-frequency sub-band; and
performing combination on the filter parameter of each sub-band, and using a combined filter bank parameter as the first hearing assistance policy for the target object.

10. The method according to claim 9, wherein
the first hearing test result comprises a hearing threshold of the target object in each sub-band; and
the determining, in descending order of frequencies of a plurality of sub-bands in an auditory frequency range, a filter parameter of each sub-band based on the first hearing test result comprises:
obtaining a gain value of each sub-band based on the hearing threshold of the target object in each sub-band and a prescription formula; and
determining, in descending order of frequencies, the filter parameter of each sub-band based on the gain value of each sub-band.

11. The method according to claim 10, wherein
the auditory frequency range comprises N sub-bands, N being an integer greater than 1; and
the determining, in descending order of frequencies, the filter parameter of each sub-band based on the gain value of each sub-band comprises:
substituting a gain value of the N^{th} sub-band into a filter function for calculation to obtain a filter parameter of the N^{th} sub-band; and
determining a filter parameter of an i^{th} sub-band based on a difference between a gain value of the i^{th} sub-band and a frequency response of a filter of an (i+1)^{th} sub-band at the it sub-band; a value range of i meeting 1≤i≤N−1, and a frequency of the (i+1)^{th} sub-band being greater than a frequency of the it sub-band.

12. The method according to claim 1, wherein the method further comprises:
amplifying the first audio signal according to at least one gain curve, to obtain a second test audio signal of at least one volume;
generating a second hearing test result of the target object in response to a feedback operation on the second test audio signal; and
transmitting a second hearing assistance policy to the audio device, the second hearing assistance policy being obtained by adjusting the first hearing assistance policy according to the second hearing test result, and wherein the second hearing assistance policy is configured to be applied to the audio device to output a second audio signal adapted to the second hearing test result to replace the first audio signal.

13. The method according to claim 12, wherein the generating a second hearing test result of the target object in response to a feedback operation on the second test audio signal comprises:
displaying a second adjustment control and a plurality of fifth feedback controls in the human-computer interaction interface, each fifth feedback control being corresponding to a pitch;
adjusting a gain of the currently outputted second test audio signal in response to a trigger operation on the second adjustment control;
sequentially outputting a plurality of second test audio signals, and recording a fifth feedback control triggered in the plurality of fifth feedback controls each time the second test audio signal is outputted; and
generating the second hearing test result of the target object based on pitches respectively corresponding to the plurality of the second test audio signals and fifth feedback controls respectively triggered in a plurality of test processes.

14. The method according to claim 13, wherein during the displaying a second adjustment control and a plurality of fifth feedback controls in the human-computer interaction interface, the method further comprises:
displaying a plurality of volume controls in the human-computer interaction interface, a volume represented by a volume control in a selected state being applied as a volume used during outputting of the second test audio signal.

15. The method according claim 12, wherein
the second hearing test result comprises a pitch incorrectly recognized by the target object; and
before the transmitting a second hearing assistance policy to the audio device, the method further comprises:
adding, according to the pitch incorrectly recognized by the target object, an adjustment amount of the pitch incorrectly recognized by the target object in the first hearing assistance policy, to obtain the second hearing assistance policy.

16. The method according to claim 12, wherein the method further comprises:
determining a gain factor of the first audio signal according to characteristic information of the target object;
generating the at least one gain curve according to the hearing parameter comprised in the first hearing test result, the gain factor, and the prescription formula, each gain curve being corresponding to a volume, and the hearing parameter comprising at least one of the following: the hearing threshold of the target object in each sub-band in the auditory frequency range, and the pain threshold of the target object in each sub-band in the auditory frequency range; and
performing interpolation processing on one of the at least one gain curve through frequency band mapping, so that a number of sub-bands of the gain curve is consistent with a number of channels of a filter bank.

17. The method according to claim 12, wherein the method further comprises:
respectively adjusting the second audio signal based on a plurality of candidate hearing sense adjustment policies, to correspondingly obtain a plurality of third test audio signals;
generating a third hearing test result of the target object in response to a feedback operation on the plurality of third test audio signals; and
transmitting a third hearing assistance policy to the audio device, the third hearing assistance policy being obtained by adjusting the second hearing assistance policy according to the third hearing test result, and being applied to the audio device to output a third audio signal adapted to the third hearing test result to replace the second audio signal.

18. The method according to claim 17, wherein
the third hearing test result comprises a preferred hearing sense of the target object; and
the generating a third hearing test result of the target object in response to a feedback operation on the plurality of third test audio signals comprises:
displaying a plurality of sixth feedback controls in the human-computer interaction interface, each sixth feedback control being corresponding to a hearing sense; and
sequentially outputting a plurality of third test audio signals in one-to-one correspondence with the plurality of sixth feedback controls, and determining a hearing sense corresponding to a sixth feedback control triggered in the plurality of sixth feedback controls as the preferred hearing sense of the target object.

19. An audio signal processing apparatus, the apparatus comprising:
a memory, configured to store an executable instruction; and
at least one processor, configured to execute the executable instruction stored in the memory, to implement:
displaying a hearing test control in a human-computer interaction interface;
outputting a first test audio signal in response to a trigger operation on the hearing test control;
displaying a first hearing test result of a target object in response to a feedback operation on the first test audio signal; and
transmitting, to an audio device in response to a configuration operation on the audio device, a first hearing assistance policy generated according to the first hearing test result, wherein the first hearing assistance policy is configured to be applied to the audio device to output a first audio signal adapted to the first hearing test result.

20. A non-transitory computer-readable storage medium, storing an executable instruction, when executed by at least one processor, causing the at least one processor to perform:
displaying a hearing test control in a human-computer interaction interface;
outputting a first test audio signal in response to a trigger operation on the hearing test control;
displaying a first hearing test result of a target object in response to a feedback operation on the first test audio signal; and
transmitting, to an audio device in response to a configuration operation on the audio device, a first hearing assistance policy generated according to the first hearing test result, wherein the first hearing assistance policy is configured to be applied to the audio device to output a first audio signal adapted to the first hearing test result.
